SEQUENCE LISTING (21 sequences)

SEQ ID NO: 1 - protein, 832 aa, Thermus aquaticus [sequence omitted]

SEQ ID NO: 2 - protein, 831 aa, Artificial Sequence
  Synthetic M1 polymerase M747K
MRGMLPLFEPKGRVLLVDGHHLAYRTFHALKGLTTSRGEPVQAVYGFAKSLLKALKEDGDAVIWFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLMALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRAMSLEVAEEIARLEAEVFRLAGHPFNLNSRDQLERVLFDELGLPAIGKTEKTGKRSTRAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDFHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKGVREAAERMAFNKPVQGTAADLMKLAMVKLFPRLGEMGARMLLQVHDELVLEAPKERAEAVARLAKEVMEGVYPLAVPLEVEVGIGEDWLSAKE

SEQ ID NO: 3 - protein, 832 aa, Thermus thermophilus
EAMLPLFEPKGRVLLVDGHHLAYRTFFALKGLTTSRGEPVQAVYGFAKSLLKALKEDGYKAVFWFDAKAPSFRHEAYEAYKAGRAPTPEDFPRQLALIKELVDLLGFTRLEVPGYEADDVLATLAKKAEKEGYEVRILTADRDLYQLVSDRVAVLHPEGHLITPEWLWEKYGLRPEQWVDFRALVGDPSDNLPGVKGIGEKTALKLLKEWGSLENLLKNLDRVKPENVREKIKAHLEDLRLSLELSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRLDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGMGEDWLSAKG

SEQ ID NO: 4 - protein, 834 aa, Thermus sp. Z05
MKAMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGFEADDVLATLAKKAEREGYEVRILTADRDLYQLVSDRVAVLHPEGHLITPEWLWEKYGLKPEQWVDFRALVGDPSDNLPGVKGIGEKTALKLLKEWGSLENILKNLDRVKPESVRERIKAHLEDLKLSLELSRVRSDLPLEVDFARRREPDREGLRAFLERLEFGSLLHEFGLLEAPAPLEEAPWPPPEGAFVGFVLSRPEPMWAELKALAACKEGRVHRAKDPLAGLKDLKEVRGLLAKDLAVLALREGLDLAPSDDPMLLAYLLDPSNTTPEGVARRYGGEWTEDAAHRALLAERLQQNLLERLKGEEKLLWLYQEVEKPLSRVLAHMEATGVRLDVAYLKALSLELAEEIRRLEEEVFRLAGHPFNLNSRDQLERVLFDELRLPALGKTQKTGKRSTSAAVLEALREAHPIVEKILQHRELTKLKNTYVDPLPGLVHPRTGRLHTRFNQTATATGRLSSSDPNLQNIPIRTPLGQRIRRAFVAEAGWALVALDYSQIELRVLAHLSGDENLIRVFQEGKDIHTQTASWMFGVSPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPHLREMGARMLLQVHDELLLEAPQARAEEVAALAKEAMEKAYPLAVPLEVEVGIGEDWLSAKG

SEQ ID NO: 5 - DNA, 18 bp, Artificial sequence
  Synthetic primer/probe
gtaacgcgctttcccacc

SEQ ID NO: 6 - DNA, 21 bp, Artificial sequence
  Synthetic primer/probe
tgtgggcattcagtctggatc

SEQ ID NO: 7 - DNA, 24 bp, Artificial Sequence
  Synthetic primer/probe
ctgatcaattccacagttttcgcg

SEQ ID NO: 8 - DNA, 25 bp, Artificial sequence
  Synthetic primer/probe
ctgttaatgggttcttagttgttgg

SEQ ID NO: 9 - DNA, 25 bp, Artificial Sequence
  Synthetic primer/probe
caaaatatggtccagttgatggaga

SEQ ID NO: 10 - DNA, 30 bp, Artificial Sequence
  Synthetic prime/probe
gttgtaattgtttttgttcctggttcacct

SEQ ID NO: 11 - DNA, 21 bp, Artificial Sequence
  Synthetic primer
cctttttgaactcttcaaacc

SEQ ID NO: 12 - DNA, 19 bp, Artificial Sequence
  Synthetic primer
tctcaaagaaacacgtgcc

SEQ ID NO: 13 - DNA, 26 bp, Artificial Sequence
  Synthetic primer
actcaaggkaacactgtgggcgtaag

SEQ ID NO: 14 - DNA, 18 bp, Artificial Sequence
  Synthetic primer
tgccacgagtyagagcca

SEQ ID NO: 15 - DNA, 18 bp, Artificial sequence
  Synthetic primer
gtgggtccgagagtytgc

SEQ ID NO: 16 - DNA, 22 bp, Artificial Sequence
  Synthetic primer
tccttctcccagtcagccccac

SEQ ID NO: 17 - DNA, 27 bp, Artificial Sequence
  Synthetic primer
agagttcaatatgttrttagcctccat

SEQ ID NO: 18 - DNA, 22 bp, Artificial Sequence
  Synthetic primer
ttccccartcactctyctttgt

SEQ ID NO: 19 - DNA, 26 bp, Artificial Sequence
  Synthetic primer
caccggayackgcagctgactcagaa

SEQ ID NO: 20 - DNA, 28 bp, Artificial sequence
  Synthetic primer
gatacactgggatgactctttgccgaac

SEQ ID NO: 21 - DNA, 23 bp, Artificial sequence
  Synthetic primer
  misc_feature (10)..(12): locked in the 3?-endo confirmation
gagtaggagtttgtgaaagtgtc

(19) United States
(12) Patent Application Publication
(10) Pub. No.: US 2024/0263255 A1
(43) Pub. Date: Aug. 8, 2024

(54) METHODS AND COMPOSITIONS TO DETECT MICROBES AND VIRAL PARTICLES

(71) Applicant: BG Research Ltd, Cambridgeshire (GB)

(72) Inventors: Nelson Nazareth, Cambridgeshire (GB); David Edge, Cambridgeshire (GB); Adam Tyler, Cambridgeshire (GB)

(21) Appl. No.: 17/264,370

(22) PCT Filed: Jul. 30, 2019
(86) PCT No.: PCT/GB2019/052133

§ 371 Date: Jan. 29, 2021

(30) Foreign Application Priority Data
Jul. 31, 2018 (GB) 1812428.9

(51) Int. Cl. C12Q 1/70; C12Q 1/6806; C12Q 1/6844; C12Q 1/689

(52) U.S. Cl. CPC C12Q 1/701; C12Q 1/6844; C12Q 1/689; C12Q 1/6806

(57) ABSTRACT

The present invention relates to rapid, safe and accurate methods of preparing samples that comprise microbial or viral nucleic acids for amplification and which can be used to detect the presence of highly pathogenic viruses such as Ebola. The methods involve heating the sample in the presence of a reagent that comprises a solvent and a detergent. Reagents and compositions are also provided.

### CROSS-REFERENCE TO RELATED APPLICATIONS

This application is a National Phase application under 35 U.S.C. 371 of PCT/GB2019/052133, filed Jul. 30, 2019, and claims the benefit of and priority to G.B. Application No. 1812428.9, filed Jul. 31, 2018, the disclosures of which are hereby incorporated herein by reference in their entirety.

### REFERENCE TO SEQUENCE LISTING

The Sequence Listing submitted Apr. 26, 2024, as a text file named “BGRBD_P69689US_371. txt,” created on Apr. 26, 2024 and having a size of 32,357 bytes is hereby incorporated by reference.

### FIELD OF INVENTION

The present invention relates to the detection of microbes and viral particles, in particular the detection of microbial or viral particles in crude samples.

### BACKGROUND

There is a requirement for rapid, simplified and relatively low cost molecular diagnostic testing, for example for the diagnosis of microbial or viral infections, to be used at the point of need in remote areas. A pertinent recent example would be the Ebola outbreak in the DRC in 2014 and the current ongoing outbreak in the region. Key in these environments is the ability to rapidly triage patients, such that those genuinely infected can be segregated from those suffering fevers from other causes such as Malaria. As viral haemorrhagic fevers like Ebola are so contagious, it is vital to correctly stratify the patients rapidly in a field hospital setting. Equally important is the absolute requirement to maximise the safety of the operator by minimising exposure to the infected patient blood normally used for molecular testing in this context. Pathogen diagnostics are usually performed following having performed a nucleic acid extraction of the sample, in order to purify any target pathogen nucleic acids, and which is the main risk point for lab workers. Therefore, a simple, biosafe method which uses minimal laboratory facilities and does not require skilled operators is needed.

Typically, molecular methods for screening for the presence of high containment level pathogens such as Ebola requires additional levels of biosecurity in order to protect the operator performing the test. The first step of screening for these viral haemorrhagic fevers is the taking of a venous draw of blood from the patient. The individual taking the sample will typically be wearing full personal protection which includes multiple pairs of gloves, a protective suit and face mask. Subsequent to taking the blood sample the outside of the collection vessel is sterilised by dunking in bleach and then a virucide, for example guanidium isothiocyanate, to render the virus non-infectious before a nucleic acid extraction takes place. These known methods comprise multi-step procedures and hence require trained users and access to a laboratory.

The typical volumes used in a venous draw for such testing are 3-6 ml and as such the risk of exposure to the pathogen is high when taking into account that multiple liquid transfers and the removal of caps must take place. If a smaller volume, such as a smaller volume of blood could be directly processed then this risk of exposure could be minimised.

If a smaller volume of sample, for example of blood is to be processed, in order to maximise operator safety, then there is increased onus placed on the requirement for sensitivity. The WHO R&D blueprint (https://www.who.int/blueprint/en/) defines a level of 3,000 virions/ml of whole blood as being a suitable level of detection for low cost diagnostics for the developing world. This would make possible the detection of a wide range of diseases including HIV and Hepatitis C, and including the viral haemorrhagic fevers described above (Ebola, Lassa, Marburg, Rift Valley fever, Crimean Congo fever, Nipah, Yellow fever, Dengue and others). The figure of 3,000 virions/ml would then provide the requirement for lower limit of detection and give guidance on the volume of blood required to be directly added into the reaction, since 3,000/ml is 3 viral targets per microlitre.

Improvements to current methods are required to allow safer detection of pathogens, particularly in instances where access to safety equipment is limited, such as in certain third-world countries, which coincide with a high incidence of some of the most deadly pathogens.

Blood is an easily accessible and commonly used source of potential pathogens that is routinely used in diagnosis.

Pathogens are routinely detected via PCR based diagnostics, including rtPCR. However, PCR is typically inhibited by the presence of whole blood, both in terms of the enzymatic amplification process and the optics used to detect the presence of the PCR product in some forms of PCR, such as rtPCR. Whole blood contains inhibitors such as haem, iron, immunoglobulins and others that inhibit the polymerase enzymes required to perform PCR or the reverse transcriptases necessary to amplify from RNA targets. Similarly, the fluorophores used in the real-time process are quenched by substances like haem in the blood and blood itself has its own absorbance and emission spectra, the net result being that real-time PCR is considered to be unreliable in the presence of high concentrations of blood. The onus on sensitivity (particularly when using low volumes of blood) means that the blood must, by necessity, form a greater percentage of the total reaction volume in the PCR and will reach the point at which it is no longer possible to perform real-time PCR due to the opacity of the reaction.

In view of the above, PCR based diagnostics using blood samples typically require multiple steps, for instance in removing the red blood cells so that the plasma or serum could be taken into the PCR reaction, extracting the pathogen nucleic acid so that PCR can be performed in the absence of any inhibitors present in the lysate, and/or in extracting the resultant PCR product so that detection can be performed in the absence of the red blood cells. These multiple steps require lab equipment, a bio-safe environment and safety equipment to ensure that clinical practitioners and laboratory staff are not infected, meaning that detection is not simple, and is not suited for rapid field-based detection of pathogens.

It is a well established fact that PCR is inhibited by the presence of whole blood, there are very few peer reviewed papers on direct from blood QPCR as only very low amounts of blood can be added to a reaction before the process is completely optically inhibited. The amount of blood that can be added and the relatively small volume of the described methodologies (<50 ul) means that direct from blood pathogen detection has been very infrequently described. For standard QPCR Minogue et al (Minogue, Timothy D et al. “Cross-institute evaluations of inhibitor-resistant PCR reagents for direct testing of aerosol and blood samples containing biological warfare agent DNA” Applied and environmental microbiology vol. 80, 4 (2014): 1322-9.) described spiking spores directly into whole blood, but this was only up to 4% and critically does not describe any spinning step. There is even less in the literature concerning direct from blood reverse transcription QPCR, since there is even less literature surrounding the use of reverse transcriptases in the presence of blood. Those that do exist are centred around parasites like malaria, where large number of ribosomal RNAs can be used as a PCR target, for example there can be tens of thousands of ribosomal RNA transcripts per parasite (Taylor B J, Lanke K, Banman S L, et al. A Direct from Blood Reverse Transcriptase Polymerase Chain Reaction Assay for Monitoring Falciparum Malaria Parasite Transmission in Elimination Settings. Am J Trop Med Hyg. 2017:97(2):533-543), or viruses like Ebola where the viral titre can be in the millions per ml of whole blood (Kavit Shah, Emma Bentley, Adam Tyler, Kevin S Richards, Ed Wright, Linda Easterbrook, Diane Lee, Claire Cleaver, Louise Usher, Jane E Burton, James Pitman, Christine B Bruce, David Edge, Martin Lee, Nelson Nazareth, David A Norwood, Sterghios Athanasios Moschos. Field-deployable, Quantitative, Rapid Identification of Active Ebola Virus Infection in Unprocessed Blood. Chem. Sci., 2017; DOI: 10.1039/C7SC03281A). The inhibition of PCR by whole blood has been shown to be multi-factorial, caused by the presence of iron containing Haem compounds, immunoglobulins and simply due to the presence of competing cations in the blood such as calcium (Sidstedt, Maja et al. “Inhibition mechanisms of hemoglobin, immunoglobulin G, and whole blood in digital and real-time PCR” Analytical and bioanalytical chemistry vol. 410, 10 (2018): 2569-2583).

Methods of lysing cells, such as blood cells, are known, for instance from WO2011157989. However, the method of WO2011157989 requires high amounts of energy to allow the required freezing and thawing cycles. Such a method is not suitable for field-based detection of pathogens using battery operated PCR machines.

WO2016139443 discloses methods for performing PCR directly on a blood sample. However, this method is restricted to the use of relatively low quantities of blood in each PCR reaction, meaning that the sensitivity is limited, and is also restricted to particular excitation and emission wavelengths. These wavelengths do not correlate to the majority of commonly used fluorophores, though suitable fluorophore combinations do exist such as CY5-BHQ2, HiLyte647-QXL607, limiting the potential for multiplexing.

In view of this, single-step, closed-tube PCR based methods of pathogen detection, particularly from blood samples, that are cheap, simple and suitable for field use where access to electricity is restricted, are not available. Any instruments or assays capable of processing whole blood, such as the BioFire or SmartCycler require upstream processing and by their nature are more complex and hence expensive per test, by virtue of their absolute requirement for automating the nucleic acid extraction process.

BG Research has previously described methods for the direct lysis of cells and viruses from crude samples, WO2011157989 Kavit Shah et al. Field-deployable, Quantitative, Rapid Identification of Active Ebola Virus Infection in Unprocessed Blood. Chem. Sci., 2017; DOI: 10.1039/C7SC03281A), and an associated optical system that makes possible the performance of multiplexed real-time PCR in the presence of whole blood, WO2016139443.

The method described in WO2011157989 results in ice crystal damage (in the case of viruses), osmotic shock as the cell thawed (in the case of fungi) and as a consequence of both for more complex targets (such as spores and bacteria), rendering the organisms detectable. However, the process of freezing adds time to the method and, since in the field these machines run on battery power, the freeze/thawing is a significant drain on power. Accordingly the present invention provides reagents and methods that make possible the in-field diagnostic detection of patients infected with diseases like Ebola, reducing the time to detection, exposure of the operator of the pathogen and removing the requirement for trained operators and the establishment of a laboratory in the outbreak zone.

It will be clear then that the present invention has particular utility in the direct detection of pathogens direct from crude samples in response to emerging outbreaks of disease. The invention is applicable to a wide range of crude sample types and pathogen families. Further, it is possible to rapidly develop new assays to the methodology to meet the requirement for responding to outbreaks of newly emerging disease, i.e. optimise the method and reagent to suit the detection of any particular pathogen. As the technique is based around the well proven RT-QPCR process, the key optimisation step is determining the thermal process, which in combination with the described reagent, can lyse and render amplifiable any newly encountered pathogen. However, it will be clear from this specification that certain embodiments of the invention are expected to require no optimisation and can be used as described to detect infection of an emerging pathogen.

The applicants have discovered that by using the methods and reagents described herein it is possible to greatly increase the amount of crude sample, for example the amount of blood that can be added to a reaction. In the case of whole blood this amount is in excess of 35%. This has the combined benefits of increasing diagnostic sensitivity but also allowing the final volume of the reaction to be minimised, thus permitting more rapid thermal cycling and thereby reducing the time to detection which is vital in a point of care field setting. Additionally, it reduces the cost per test significantly by reducing the total reaction volume and hence proportionally reducing the cost of the reagents.

A number of blood borne viral infections are found in remote, resource poor environments, including Lassa, CCHF, Ebola etc, and minimum time to detection, ease of use and cost per test are all vital. The WHO R&D blueprint states that simplified molecular diagnostics for the developing world must have a sensitivity of 3000 virions/ml and a cost per test equivalent to antibody-based approaches. The present invention achieves this, increasing the sensitivity to below 1000 virions/ml and reducing the cost to that of lateral flow immunodiagnostics. Assays using the described method have been shown capable of detecting as few as 15 virions per reaction, so assuming that 15 μl of blood has been added this would be a final sensitivity of 1000 virions/ml.

Where the sample is a blood sample, the applicants have discovered that some reagent mixtures, based on variables such as pH and the presence of adjuncts, can be more denaturing to the blood. The consequence of that is that the optical system described in WO2016139443 (which describes a high powered laser based spectrometry based approach for multiplexed detection in the presence of whole blood) is no longer in those circumstances able to detect real-time PCR signals in blood concentrations as high as the 13% maximum stated in that application (Kavit Shah, Emma Bentley, Adam Tyler, Kevin S Richards, Ed Wright, Linda Easterbrook, Diane Lee, Claire Cleaver, Louise Usher, Jane E Burton, James Pitman, Christine B Bruce, David Edge, Martin Lee, Nelson Nazareth, David A Norwood, Sterghios Athanasios Moschos. Field-deployable, Quantitative, Rapid Identification of Active Ebola Virus Infection in Unprocessed Blood. Chem. Sci., 2017; DOI: 10.1039/C7SC03281A). The reason for this reduced performance is that in a more denaturing reagent, the blood turns from a red liquid into a “brown” colloidal suspension of denatured protein which increases the opacity of the liquid. At higher percentages of blood a dark brown colloidal suspension is formed that completely prevents the collection of optical data. Although the applicants have been able to formulate reagents capable of performing reverse transcript quantitative PCR (RT-QPCR) in the presence of as much as 40% whole blood the process is not viable as optical data can no longer be discerned.

### BRIEF SUMMARY OF THE INVENTION

The invention provides improved reagents and methods for the processing and detection of microbes and viral particles in a sample. The invention is particularly advantageous when the sample is a crude sample, such as whole blood. The method of the invention typically involves a processing step in which the viral or microbial nucleic acid becomes amplifiable by, for example, a standard RT-PCR reaction, a subsequent amplification step where any target nucleic acid is amplified, and a detection step in which the amplified nucleic acid is detected. The method can be used with any fluorophore or suitable amplicon-detecting dye or real-time PCR chemistry known in the art, since any excitation and detection wavelength can be used. This renders an improvement in the number of target nucleic acid targets, in this case pathogens from whole blood, that can concurrently be screened for. The present method also does not require a nucleic acid extraction step or centrifugation like some previous methods, meaning that the present method is much simpler, requiring less laboratory equipment and less handling of the samples.

In some embodiments, the methods also involve multiple rounds of reverse transcription (RT). This is shown to greatly enhance the sensitivity of the reaction and can detect the presence of very low abundance viruses directly from crude samples such as blood.

The method can be combined with the teachings of PCT/GB2019/051156 to be used as a system for the direct detection of viral pathogens in whole blood, effectively making plasma in a closed tube, lysing the pathogen contained therein and then directly performing RT-QPCR on the target pathogen of interest, following the centrifugation step an optional freeze-thaw cycle could be performed (EP2585581), hence rendering a wide range of target pathogens directly amplifiable.

### DETAILED DESCRIPTION OF THE INVENTION

In one aspect, the invention provides a method of preparing a sample for the direct amplification of target microbial or viral nucleic acid that may be present in the sample. The inventors have found that whilst heating samples in some instances may be sufficient to obtain nucleic acid that is accessible to the PCR reagents and that can be amplified, heating alone is not reliable and cannot be generally applied widely to different target microorganisms or viruses. This is particularly the case where the sample is a crude sample, for example a crude sample obtained from a subject, such as a blood sample, that often itself can comprise compounds that inhibit the PCR reaction.

Accordingly, in one aspect, the invention provides a method of preparing a sample obtained from a subject that may contain one or more microbes or viral particles for the direct amplification of the microbial or a viral particle nucleic acid by a polymerase,

wherein the method comprises heating the sample in a vessel to a temperature of at least 70° C. in the presence of a reagent that comprises a detergent, a solvent and one or more nucleic acid polymerases.

Present methods of pathogen diagnostics are usually performed following a nucleic acid extraction, for example a salt/alcohol extraction, of the sample, in order to purify any target pathogen nucleic acids. This generally places a requirement for the use of dedicated laboratory facilities and trained molecular biologists and as such is not suited to responding to outbreaks of newly emerging pathogens, particularly in countries or regions in which access to sophisticated laboratory equipment is limited. Nucleic acid extraction is performed for two reasons, firstly to ensure that the pathogen is disrupted in order to release the target nucleic acid and secondly in order to remove inhibitors present in the sample that may negatively impact the performance of the molecular diagnostic assay, such as PCR inhibitors.

In order to perform direct diagnostics it is therefore necessary to accomplish this duality of function in a single closed tube process. The pathogen must be lysed/nucleic acid rendered detectable, and the reagent must be capable of performing the amplification process in the presence of sufficient crude sample to enable a diagnostic detection to take place. For example in the case of Ebola an individual presenting with symptoms will have a viral load of in excess of 1e6 virions/ml of blood and that viral load is directly related to the severity of disease and attendant risk of fatality, this means that each microlitre of whole crude blood would contain 1000 genome targets and would have diagnostic utility (Hartley M A, Young A, Tran A M, Okoni-Williams H H, Suma M, et al. (2017) Predicting Ebola Severity: A Clinical Prioritization Score for Ebola Virus Disease. PLOS Neglected Tropical Diseases 11(2): e0005265). The WHO defines a sensitivity level of 3000 virions/ml as being suitable for simple, low cost, tests for use in remote poor regions. As an example, this would be sufficient to detect important diseases such as hepatitis C and HIV in a developing world context where the viral load can be below 10,000 virions/ml. As a result in these cases it is necessary to process significant volumes of crude sample, since each microlitre will only contain in the region of 3 genome targets. The preferred sample type for this methodology is whole blood when the goal is detecting the presence of viral pathogens, and commonly serum and plasma are the sample types used by molecular biology laboratories. Plasma and serum are commonly used because they avoid the inhibitory compounds that can come through the extraction process and hence generate false negative results. A disadvantage of using serum and plasma is that the centrifugation used can reduce the viral titre in the sample (Klungthong C, Gibbons R V, Thaisomboonsuk B, et al. Dengue virus detection using whole blood for reverse transcriptase PCR and virus isolation. J Clin Microbiol. 2007; 45(8): 2480-5). Therefore, the ideal approach for direct detection of microbial or viral pathogens that are present in a sample, such as blood, is to directly add the sample, such as a volume of whole blood, into a sealed reaction vessel, to maximise biosafety and sensitivity, ensuring that the nucleic acid present in any microbes or viruses becomes amplifiable, and performing direct amplification. By amplifiable we include the meaning that the nucleic acid that is inside the microbe or virus is made accessible to the PCR components, such as primers and polymerase(s); and that the influence of any amplification inhibitors is sufficiently mitigated so that the nucleic acid present in the environment of the vessel is able to be amplified, for example is able to be amplified by PCR, or reverse transcription PCR (RT-PCR), or real time (rt) PCR (rtPCR) or quantitative (q) PCR (qPCR), or reverse transcription quantitative-PCR (RT-qPCR).

By “direct amplification” we include the meaning that following the method of preparing the sample, amplification, for example PCR or RT-PCR or RT-qPCR may be performed on the sample as is, i.e. no further processing of the nucleic acid is required, and providing that all of the components needed for the amplification are already present, or are added to the sample, amplification can occur. By direct amplification we also include the meaning that no components need removing from the sample following the processing, i.e. it is not necessary to, for example, centrifuge the sample and remove the pelleted material or another fraction that may be considered to comprise amplification inhibitors. In one preferred embodiment, the vessel comprises all components necessary for amplification so that once the sample has been added to the vessel, amplification can proceed. Once the sample has been added, in some embodiments such as those where the vessel comprises all components needed for amplification and detection, the vessel can be sealed and need not ever be opened again. As the method prepares the sample for the direct amplification of the viral or microbial nucleic acid, the methods of the invention have particular utility in the detection of highly pathogenic microbes and viruses. In these circumstances, it is critical to reduce the exposure of the clinician/field worker to the pathogen as much as possible. The present invention provides that the clinician/field worker need not be exposed to any pathogenic material once the sample has been taken and added to the vessel.

In some embodiments the method is a closed-tubed method of preparing the sample. The skilled person will understand what is meant by a closed-tubed method and typically requires that once the sample has been added to the vessel, along with any necessary components for the method of preparing, or downstream amplification and detection steps, the vessel is closed, for example by closing a cap or sealing a lid onto the vessel, and the cap or lid is not opened again. For example in some embodiments of the closed tube method no further material is added or removed from the vessel, for example the nucleic acid is not extracted or in any way purified.

Accordingly, in one embodiment the microbial or viral nucleic acid is not extracted, for example the microbial or viral nucleic acid is not precipitated with an alcohol, for example is not precipitated with ethanol. The skilled person will understand what is meant by nucleic acid extraction and typically is the purification of nucleic acid from its environment. Accordingly, in this embodiment, the nucleic acid is not removed from its original environment, i.e. is not removed from the other components of the sample and reagent. In this and other embodiments the nucleic acid is not purified, i.e. is not separated from the other components of the cell or virus.

In the same or different embodiment, no part of the sample or reagent is removed from the vessel at any stage in the preparing step, for example no part of the sample or reagent is removed from the vessel:
a) prior to said heating; and/or
b) following said heating.

In one embodiment, once the sample is added to the vessel, no material is removed from the vessel. In a further embodiment, no material is removed from the vessel during preparing, amplification and detection. In such embodiments, a vessel may be used that comprises an irreversible lock so that once the sample has been added and the vessel is sealed, the vessel cannot be opened again. Such vessels are described in PCT/GB2019/051156. In one embodiment, the vessel is considered to be a bio-secure vessel. The skilled person will appreciate what is meant by the term bio-secure. In one embodiment bio-secure means that none of the pathogen contained within the vessel is able to escape from the vessel, and so the person handling the vessel cannot be exposed to any pathogen contained within. In one embodiment a bio-secure vessel has a lid or a cap which locks. Preferably a bio-secure vessel is made of a material that is crush proof, such as a carbon loaded polymer. In another embodiment the bio-secure vessel also has 2 points of security in sealing.

In one preferred embodiment, the vessel of the invention is suitable for use in an amplification reaction, for example a PCR reaction, optionally suitable for use in reverse transcription (RT) PCR, optionally for use in quantitative (q) PCR or RT-qPCR. Vessels suitable for use in such reactions typically have thin walls of around 0.5 to 0.8 mm in thickness and made of carbon loaded polymer. This does 2 things 1) makes it gain and lose heat faster 2) reduces lag between reaction vessel holder/tube/liquid contents.

Some known methods of preparing samples for amplification involve repeatedly freeze thawing the sample. Whilst such methods are suitable for use with the present methods, it is not necessary to freeze the sample. This is an advantage of the present invention over such methods, since freeze thawing is energy intensive, meaning that such methods are not best suitable for field use, for example in countries with poor access to mains electricity where thermal cyclers are battery powered. The present invention addresses this drawback.

Accordingly, in one embodiment, the temperature of the sample is not reduced relative to the ambient temperature, optionally wherein the sample is not frozen.

Other means to prepare samples for nucleic acid amplification involve centrifugation of the sample which can a) pull down particulate matter which interferes with amplicon detection; and b) pull down material that can comprise PCR inhibitors. However, as with the freeze thaw methods, centrifugation requires additional laboratory equipment and energy. As discussed above, centrifugation can also remove some viral or microbial matter meaning that there are fewer target nucleic acids available to act as a template in the amplification. The present invention is simpler, low energy, low-skill, and addresses issues with particulate matter interfering with the light path of spectrophotometers and amplification inhibition. Inhibition of amplification can, to some extent, be mitigated by choosing and appropriate polymerase(s) enzyme, for example choosing an enzyme that is tolerant of inhibitors present in blood or urea or saliva, for example. The skilled person will be aware of enzymes that are tolerant of particular sample types and is also aware of how to determine the most appropriate enzyme to use with a given sample type.

Accordingly, in one embodiment the sample is not centrifuged:
a) before heating; and/or
b) following said heating.

However, there may be instances where centrifugation is appropriate, for example simply to pull sample and reagent into the vessel that may, for example, have condensed on the cap of the vessel. For example the sample may be pulse centrifuged, for example for 5 s to 15 s at speeds of 1-2000 g simply to pull down the condensation into the vessel.

Preferably, the sample is a crude sample. By a crude sample we include the meaning of a sample to which no, or minimal, additional components have been added and/or processing steps have been applied following obtaining the sample from the subject that change the composition of the sample. For example, the crude sample may be a crude biological sample, such as
a) blood, optionally whole blood;
b) urine;
c) serum;
d) plasma;
e) faeces,
f) cerebral spinal fluid;
g) a swab, optionally a swab from the eyes, ears, nose or mouth; and/or
h) eluate taken from a wash of a swab.

In some exemplary embodiments, a swab taken from a human, and animal, or an environmental surface, or another solid sample type, can be placed in water, for example 200 μl water and vortexed to release viral or microbial particles. This water can then be added to the vessel, at up to 20% of the reaction volume. Similarly, liquid environmental samples can be added straight to the vessel.

The skilled person will understand what is meant by a crude sample. In one embodiment a crude sample is a sample in which no attempt has been made to purify the target nucleic acid from its native environment, e.g. purify from the blood cell, urine sample, faecal sample, spinal fluid or swab. An eluate taken from a wash of a swab is considered to be a crude sample since the target nucleic acid at this stage is still likely associated with cellular matter or viral particles.

Since the present invention may also be applied to the detection of disease in animals, the ability to use a swab, or swab elute as a sample is important. In the veterinary field the most universally used sample type is the swab—these being taken from either the eyes, the nose or the mouth dependent upon the disease suspected. A number of virulent animal pathogens, such as Rinderpest and PPRV, have a viraemic component but for some economically important diseases there is a very limited time window that the viral pathogen can be found in the blood. However, there are a number of disadvantages to using a direct from blood approach in the remote, resource poor environments where these diseases are endemic. Firstly, the taking of a blood sample from an animal requires the input of a trained veterinarian and secondly that the virus can be found in easier to access samples that can be taken by the lay person. This application covers a method for performing direct detection of viral animal pathogens direct from swab samples taken from the mouth, eyes or nose and without recourse to nucleic acid extraction. The applicants have also identified that this can be applied to other important diseases, for example respiratory disease in humans. The swab is simply taken from the patient or animal, put into a plastic tube containing 200 μl of water and then the tube is shaken to release the viral particles from the swab. A small proportion of this liquid is then transferred directly into the reaction vessel in lieu of the blood, or other liquid sample types previously described.

A crude biological sample is considered to be any sample taken directly from an organism. Although the present invention is considered to have its main utility in the diagnosis of pathogenic infections, the crude sample may also be a crude environmental sample, in which case the “subject” of the claim can be taken to refer to the surface or origin of the environmental sample. By a crude environmental sample we include the meaning of samples such as food samples, swabs taken from the environment, such as surfaces and any other sample type that isn't taken directly from an organism (in which case it would be considered to be a crude biological sample directly taken from an organism).

The crude sample may also be a direct crude environmental sample, for example a direct sample of water from a stream or lake, a direct sample of soil or other environmental material. The sample may also be a plant sample.

The crude sample may also be a sample to which minimal processing steps have been applied following obtaining the sample, such as in the preparation of plasma and serum from whole blood, or an eluate from a wash of a swab, for example for pathogens that are not highly infectious to the operator, for example when used for the detection of veterinary pathogens in the field. The crude sample may also have had some additional components added, for example preservatives, but these are not considered to result in any purification of the nucleic acid from the sample, in other words, all of the original material in the sample remains present. In the case of a blood sample, EDTA may be added to the sample for storage, but the sample is still considered to be a crude sample. Swabs and samples may be frozen before being prepared according to the method of the invention, but preferably the method is performed immediately, or as soon as possible on the sample once it has been taken from the subject to at least minimise contamination and infection.

As discussed, the processed sample is typically used in an amplification reaction to amplify the target nucleic acid. The amplified target, or amplicon, is then detected, either following amplification or during amplification, for example using qPCR or RT-qPCR. The skilled person will understand that qPCR and RT-qPCR typically requires the use of fluorophores or other suitable nucleic acid intercalating or detecting dyes and probe chemistries.

The skilled person will understand what is meant by qPCR. In this case the reaction contains primers, or groups of primers when the target pathogen has high levels of sequence heterogeneity, such as Lassa, and a probe which is sequence specific to the target of interest. This could be a hydrolysis probe whereby the 5′ end is labelled with a fluorophore and the 3′ with a quenching moiety—during amplification the probe is hydrolysed by the enzyme and as such fluorescence increases cycle on cycle. Excitation means is provided and the resulting emission is captured through a window in the vessel, which in some embodiments is located in the cap.

In some preferred embodiments, the fluorophores or dyes are present during the processing step, for example as part of a fluorophore labelled primer or probe. The crude sample will typically comprise particulate or cellular material which in some embodiments may be considered to interfere with the excitation of fluorophores and/or capture of the emitted wavelengths and may otherwise interfere with the choice of suitable fluorophores. Known methods to circumvent these issues involve centrifugation and/or the use of particular fluorophore combinations that excite and emit at wavelengths that are not absorbed by the sample, for example are not absorbed by blood, for example far red fluorophores. However, the reagent used in the present method addresses these issues since it results in the aggregation or coagulation of the sample, such as whole blood, so that large deposits are formed which sink to the base of the vessel. The present invention is particularly suited to use with a whole blood sample, though the reagents provided will sink proteinaceous material in any sample that is rich in protein. Provided that the vessel is not substantially disturbed or agitated, the deposits remain at the base of the vessel leaving a clarified top layer which can be used for fluorophore excitation and emission. Accordingly, fluorophores or dyes that excite and emit at any wavelength can be used with the present invention, without the need for a centrifugation step. For example, the sample may be excited at a wavelength of between 300 nm and 800 nm and the emitted light may be collected at any wavelength, for example between 300 nm and 800 nm. In some embodiments the excitation wavelength used to excite the fluorophore associated with the qPCR is between 630 nm-645 nm, optionally between 633 nm-642 nm; and/or the emitted light is collected at a wavelength of between 650 nm-750 nm. In another embodiment the fluorophore is excited at a wavelength of around 475 nm and/or 635 nm; and/or the emitted light is collected at a wavelength of around 520-50 nm and 660-750 nm.

In one embodiment the PCR uses a 2 colour system with LED excitation at 475 nm and 635 nm and collection of the emission at 400-900 nm using a dual band pass filter with windows of 520-580 nm and 660-750 nm.

Exemplar fluorophores include FAM, TET, JOE, VIC, HEX, NED, PET, ROX, TAMRA, CY5. However the skilled person will appreciate that since in the present method the sample does not interfere with excitation or emission, the choice of suitable fluorophore or dye is limited only by the excitation and collection capabilities of the thermal cycler being used.

Although not required by the present invention, it is possible to perform a centrifugation step on the sample which may be beneficial in some cases. For example, by performing the centrifugation step, the crude sample, for example the blood, may be removed faster from the optical path.

It is considered preferable from a safety point of view if the sample is obtained and added directly to the vessel (which may or may not be a vessel according to PCT/GB2019/051156). The skilled person will of course appreciate that following obtaining the sample the sample may be stored for some period of time, for example at cold temperatures, prior to adding to the reaction vessel.

As stated above, the sample will typically be a biological sample, for example may be a sample taken from a mammalian animal, for example from a human, cattle, swine, cow, sheep, pig, dog, camel, horse, llama, goat, rabbit, cat, rat mouse, ferret, guinea pig, mink and other model organism. In other embodiments the sample is a sample taken from an avian species. In other embodiments the sample is a sample taken from a fish. Preferably the sample is a human sample or a sample from cattle.

The microbial or viral particle that is to ultimately be detected may be any microbial or viral particle, for example may be any of viruses, bacteria, protozoan or fungi. In preferred embodiments the microbe or virus is a pathogenic microbe or virus, for example a class 3 or 4 pathogen as classified by Classification of Biological agents, National Institute for Public Health and the Environment, RIVM Letter Report 205084002:

| | Ability to cause | | Spread to the | Effective |
|---|---|---|---|---|
| | disease in humans | Hazard to | community | prophylaxsis or |
| Classification | (virulence) | workers | (transmission) | (treatment) |
| Category 1 | Unlikely | No hazard | Not applicable | Not applicable |
| Category 2 | Likely disease | Potential hazard | Unlikely | Available |
| Category 3 | Likely severe disease | Serious hazard | Likely | Available |
| Category 4 | Severe disease | Serious hazard | High risk | Not available |

Similar definitions are also used by other countries and international organizations:

| | |
|---|---|
| WHO | Laboratory Biosafety Manual, 3rd Edition (2004) |
| PHAC | Laboratory Biosafety Guidelines, 3rd Edition (2004) |
| CDC | Biosafety in Microbiological and BioMedical Laboratories (BMBL), 5th Edition (2009) |

Note.
An extensive overview of the criteria of biological agents is presented on the website of the American Biological Safety Associaion-ABSA

The pathogen may be a mammalian pathogen, for example a human pathogen, cattle pathogen, swine pathogen, cow pathogen, sheep pathogen, pig pathogen, dog pathogen, camel pathogen, horse pathogen, llama pathogen, goat pathogen, rabbit pathogen, cat pathogen, rat pathogen, mouse pathogen, ferret pathogen, guinea pig pathogen, mink pathogen, or other model organism pathogen, or is an avian pathogen or is a fish pathogen.

In other embodiments, or the same embodiment, the pathogen is an avian pathogen. Accordingly in one embodiment the one or more microbes or viral particles may be selected from the group consisting of:
a) Viral haemorrhagic fevers selected from the group consisting of Ebola, Lassa fever, Marburg virus disease, Rift valley fever, Congo fever and yellow fever; and/or
b) Japanese encephalitis, Dengue, Zika, Chikungunya;
c) Veterinary diseases with a viraemic component, including but not limited to PPRV, FMDV, BTV, Newcastle disease, Swine Flu, BVDV
d) Malaria, HIV, viral hepatitis, soil transmitted helminth parasitic infections.
   By viraemic we include the meaning of having a blood borne component.

The present invention is considered to have particular utility in preparation of, and then subsequent amplification and detection of microbes or viruses that have RNA genomes.

In one embodiment the viral particle is an RNA viral particle. In some embodiments the RNA viral particle is selected from the group consisting or comprising: Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus

The present invention is considered to be suitable for the processing and subsequent amplification of both enveloped and non-enveloped viruses.

As described above, the sample is heated in a vessel to a temperature of at least 70° C. in the presence of a reagent that comprises a detergent, a solvent and one or more nucleic acid polymerases. In preferred embodiments no further physical manipulation of the sample is performed, or no further material is added to the vessel once the sample has been added, so the reagent that comprises the detergent and a solvent should be compatible with the one or more nucleic acid polymerases to allow the enzyme to function in downstream amplification. By compatible we mean that the reagent should substantially maintain the full maximum activity of the enzyme. For example, the enzyme should retain at least 98%, 96%, 94%, 92%, 90%, 85%, 80%, 75%, 70%, 65% 60% activity. The skilled person will understand how to determine the maximum activity of a polymerase and the relative change in activity of the polymerase when used in different reagents. For example, a real-time PCR reaction with a naked RNA template can be performed, for example using the buffer that the enzyme is supplied in can be considered to provide 100% activity. The effects of addition of various components of the reagent can be determined, for example by adding tween and/or glycerol and measuring the performance of the polymerase. In some embodiments the reagent is compatible with the polymerase if amplification occurs with the polymerase in the presence of the reagent, i.e. amplification of any amount. Accordingly, in one embodiment the reagent is compatible with the one or more polymerases. Practically it is considered appropriate if the Ct of a reaction in the presence of a particular reagent component is within 0.5 of the Ct of the reaction in the absence of the reagent component.

The skilled person will understand what components of the reagent will be compatible with a particular polymerase.

Further, in the same or alternative embodiment, the reagent is compatible with the reagent in which the one or more polymerases is supplied. The skilled person will understand that typically commercially available polymerases are supplied in liquid form and so are already associated with various components. It is considered that in some circumstances there may be an interaction between the reagent that the polymerase is supplied in and the reagent of the invention which may reduce or inhibit the activity of the polymerase. As above, the skilled person will be aware of this and can take steps to ensure that the reagent of the invention is compatible with the reagent of the enzyme. In other embodiments, the polymerase is supplied lyophilised in which case the polymerase is not already associated with any reagents and so this compatibility is not an issue. Preferably the polymerase is supplied lyophilised in the vessel to which the sample is added.

The skilled person will be aware that polymerases can also be supplied in a pure enzyme liquid phase, i.e. where the enzyme is not already in any reagent- or is supplied without any associated storage buffers.

Since, in some instances, the target nucleic acid is RNA an additional issue is the thermal stability of the released RNA itself. The skilled person is aware that RNA degrades in the presence (Valles S M, Strong C A, Buss E A, Oi D H. Non-enzymatic hydrolysis of RNA in workers of the ant Nylanderia pubens. J Insect Sci. 2012; 12:146. doi: 10.1673/031.012.14601) of divalent cations in a basic solution. As a result, if the method of preparing described here is used with a standard Taq polymerase buffer which is typically based on Tris and 4M MgCl₂, any released RNA would be rapidly degraded and the target RNA rendered non-amplifiable. The skilled person will understand that the reagent may therefore comprise one or more buffering agents. The reagent may comprise any buffering agent and the skilled person is aware of such agents. However, in one embodiment the reagent does not comprise Tris.

The applicants have developed buffers that are substantially pH neutral at the temperature that the sample is heated to, i.e. above 70° C., for example the capsid Tm where the target nucleic acid is viral nucleic acid, and that become neutral above those temperatures. The skilled person will understand that it is the PKA of the buffer that indicates whether the buffer will be at a neutral pH at a given temperature. For example the PKA of bicine is 0.018 pH/C. Further, the system buffers divalent cations such that the free concentrations are minimised. In some embodiments the reagent is therefore based on bicine or tricine—a typical formulation might be 50 mM bicine/tricine, 3.5 mM MgCl₂, 115 mM potassium acetate and adjusted to pH 8.2 at 25° C. This reagent will be essentially neutral at temperatures above 70° C., for example at the capsid Tm (capsid denaturation point), and as such there will be no excess hydroxyl ions to attack the RNA and yet the pH will be at the physiological requirement for the enzyme at the reverse transcription/amplification step which will be performed in the range of 55 C to 65 C, pH 7.4-7.6.

Accordingly one embodiment provides a reagent that has a pH of around 6.5-7.5 at a temperature of between around 70° C.-100° C., optionally between 72° C. and 98° C., 74° C. and 96° C., 76° C. and 94° C., 78° C. and 92° C., 80° C. and 90° C., 82° C. and 88° C., or to between 84° C. and 86° C.; optionally wherein the reagent is at pH 7.45 at 70 C and/or is at a pH of 7.0 at 95° C.

Accordingly, in a preferred embodiment the reagent minimises RNA degradation and an example of suitable components are given above.

Since divalent cations are typically required as cofactors for polymerase activity, in some embodiments it is considered important if the reagent buffers divalent cations. Suitable buffers include bicine and tricine.

In another embodiment, a bicine based buffer is preferred. The concentration of bicine can be any concentration. Accordingly, in one embodiment the reagent comprises bicine. In another embodiment the reagent comprises bicine at a concentration of between 20 mM and 70 mM Bicine, between 25 mM and 65 mM Bicine, between 30 mM and 60 mM Bicine, between 35 mM and 55 mM Bicine, between 40 mM and 50 mM Bicine, or around 50 mM Bicine. In another or the same embodiment the reagent comprises bicine at a concentration of at least 20 mM, 25 mM, 30 mM, 35 mM, 40 mM, 45 mM, 50 mM, 55 mM, 60 mM, 65 mM, 70 mM. In another or the same embodiment the reagent comprises bicine at a concentration of less than 70 mM, 65 mM, 60 mM, 55 mM, 50 mM, 45 mM, 40 mM, 35 mM, 30 mM, 25 mM, or 20 mM.

In another embodiment the reagent comprises tricine, for example comprises tricine at the concentrations indicated above.

As discussed above, the reagent requires at least one solvent and at least one detergent. In one embodiment, the solvent is glycerol. In the same or another embodiment the detergent is Tween, for example Tween 20. Accordingly in one embodiment the solvent is glycerol and/or the detergent is Tween, for example Tween 20.

In one embodiment, the reagent comprises Tween (polysorbate), for example Tween20 at a concentration of up to 0.4%, for example at a concentration of
a) at least 0.025%, 0.05%, 0.075%, 0.1%, 0.15%, 0.2%, 0.25%, 0.3%, 0.35%, 0.4%; and/or
b) less than 0.4%, 0.35%, 0.3%, 0.25%, 0.2%, 0.15%, 0.1%, 0.075%, 0.05%, 0.025%; and/or
c) between 0.025% and 0.4%, 0.05% and 0.35%, 0.075% and 0.3%, 0.1% and 0.25%, 0.15% and 0.2%;
for example wherein the concentration of tween is between 0.15% and 0.3%.

In one embodiment the reagent comprises glycerol at a concentration of 11%, or less than 11%, optionally wherein the reagent comprises less than 11%, 10%, 9%, 8%, 7%, 6%, 5%, 4%, 3%, 2%, 1%, 0.5% or less glycerol. In the same or alternative embodiment the reagent comprises glycerol at a concentration of between 0.5% and 11%, 1% and 10%, 2% and 9%, 3% and 8%, 4% and 7%, or between 5% and 6%. In the same or alternative embodiment the reagent comprises glycerol at a concentration of more than 0.5%, 1%, 2%, 3%, 4%, 5%, 6%, 7%, 8%, 9%, 10%, or more than 11%.

As discussed above, the sample is heated to at least 70° C., in the presence of one or more polymerases. Heating microorganisms such as bacteria is known to disrupt the bacteria and allow the nucleic acid to access the components necessary for amplification, such as polymerases and primers. Heating viruses is also known to allow the nucleic acid to access nucleic acid stains (the Tr) and to disrupt the protein conformation of the viral capsid which neutralises the virus TM (Walter et al 2012 J Virol Methods—the PaSTRy approach). The Tr as described in Walter et al 2012 is a lower temperature than the Tm. Despite the Tr correlating both with viral inactivation and accessibility of the nucleic acid to nucleic acid stains, the inventors of the present invention have identified that heating viruses to this lower Tr temperature is not sufficient to render the viral nucleic acid amplifiable. In fact, even heating the virus to the Tm, the temperature at which the viral proteins change conformation, (capsid denaturation temperature) is not sufficient to allow the nucleic acid to be amplified. The inventors have shown that amplification of the viral nucleic acid is only possible once the virus has been heated to the Tm of the virus in the presence of the detergent and solvent, such as tween and glycerol.

It will be appreciated that heating the sample in the presence of the polymerase is not a typical approach to take since at temperatures above 70 C most RNA dependent DNA polymerases are denatured. Accordingly, the present invention requires a thermotolerant RNA dependent DNA polymerase (where such activity is required). Examples of such enzymes are provided herein.

The inventors have found that for all viruses tested to date, the Tm (capsid denaturation temperature) of each individual virus falls within the range of 76° C.-81° C. However, it is possible that some viruses or microbes would require a higher temperature. Since the reagent of the invention is considered to be protective to any DNA or RNA, it is considered appropriate to use a higher temperature, particularly when working with a virus or microbe which has not yet been used with the method and where optimization has not been performed. However, the skilled person will understand that where possible, lower temperatures should be used to preserve both the nucleic acid and the polymerase activity. Alternatively, heating to a higher temperature means that a shorter duration of heating can be used. In some embodiments, keeping the duration of heating short is preferred, even if it means using a higher temperature.

It is considered that heating the sample to 70 C or over will cause at least some of the target viral or microbial nucleic acid to become amplifiable, e.g. to become accessible to the polymerase and other amplification components, though 70 C may not be the most optimal. The skilled person will know how to optimise the temperature, for example by performing the method of the invention as discussed herein multiple times using several different temperatures across a suitable range. However, in one preferred embodiment, the sample is heated to 93-95 C since this is suitable for all samples and no optimisation is required. The skilled person will appreciate though that for a given sample type and virus/microbe, there will be an optimal temperature and duration of heating that provides the optimal balance between accessible nucleic acid and degradation of nucleic acid.

Heating to 95 C also has the advantage that although lower temperatures may render the nucleic acid accessible to amplification components, in some instances the nucleic acid will have a secondary structure that prohibits amplification. Heating to around 95 C ensures that any secondary structure in the nucleic acid is removed.

The applicants have observed that the Tm point, the point at which the viral or microbial nucleic acid is made accessible to the amplification components, can be lowered by the addition of solvent/detergent to the amplification reagents mixture. Many solvent detergent combinations are used commercially for viral inactivation, although these were found to largely be incompatible with numerous amplification approaches. A suitable combination was found to be Glycerol at 8-11% and Tween 20 at 0.15-1% (optimally 8-9% glycerol and 0.15-0.3% Tween). For Tween the percentage added does further drop the TM point, but the applicants have also observed that the hybridisation temperature of any primers and probes, such as for the PCR process, was depressed by higher concentration of Tween that 0.4% and as a result the PCR efficiency can be observed to drop. Heat alone does not render the viral RNA amplifiable, i.e the Tm point is reached and the RNA is made accessible to intercalating dyes, yet this RNA cannot be amplified. It requires the correct thermal processing and the correct concentration of the solvent/detergent mix. This is assumed to be that the combination of glycerol and Tween assists in the denaturation of capsid protein and ensures that no lipids or proteins remain associated with the released RNA and hence render it amplifiable by the polymerase.

A preferred set of reaction conditions is as follows: 50 mM Bicine, 3.4 mM MgCl₂, 115 mM Potassium Acetate, 8% glycerol, 0.2% tween at pH 8.2 at 25° C. A range of MgCl₂ concentrations from 2.5-4 mM MgCl₂ generate amplicon but 3.4 mM MgCl₂ is optimal.

In one embodiment, the sample is heated to:
a) between around 70° C.-100° C., optionally between 72° C. and 98° C., 74° C. and 96° C., 76° C. and 94° C., 78° C. and 92° C., 80° C. and 90° C., 82° C. and 88° C., or to between 84° C. and 86° C.; and/or to
b) at least 72° C., 74° C., 76° C., 78° C., 80° C., 82° C., 84° C., 96° C., 88° C., 90° C., 92° C., 94° C., 95° C., 96° C., 97° C., 98° C., 99° C., 100° C. or more than 100° C.
   In some embodiments the sample is heated to between around 76° C.-81° C., particularly in embodiments where the target nucleic acid in the sample is viral nucleic acid and the sample is suspected of comprising viral particles. For example may be heated to at least 76.0, 76.5, 77.0, 77.5, 78.0, 78.5, 79.0, 79.5, 80.5, 81.0 or 81.5° C.

In other embodiments, for example particularly in situations where the Tm (capsid denaturation) temperature of a virus has not been determined, the sample is heated to around 93-95° C. for a period of 1-5 seconds.

In some embodiments, for example where the sample is a microbe, such as a bacteria or a fungi, the sample is heated to 90-95 C for, for example between 30 s to 60 s.

The skilled person will understand that heating a sample comprising RNA, for example viral RNA to 95 C is not a typical approach to take, since the RNA would, using prior art methods, be degraded. Similarly, heating the RNA in the presence of an RNA dependent DNA polymerase to 95 C is not a routine approach to take since the polymerase would become degraded.

Accordingly in one embodiment the sample comprises or is expected to comprise, or the target nucleic acid is, viral RNA and the polymerase has a RNA dependent DNA polymerase activity, and the sample is heated to over 90 C, for example is heated to 95 C for 1, 2, 3, 4 or 5 seconds.

The sample may be heated to the required temperature for any length of time. However, and as described above, the skilled person will understand that when working with nucleic acid, minimising exposure to high temperatures is preferable. Accordingly, in one embodiment, the sample is heated to the required temperature for:
a) between 0.5 s and 5 s, 1 s and 4.5 s, 1.5 s and 4 s, 2 s and 3.5 s, or between 2.5 s and 3 s; and/or
b) at least 0.5 s, 1 s, 1.5 s, 2 s, 2.5 s, 3 s, 3.5 s, 4 s, 4.5 s or at least 5 s; and/or
c) less than 5 s, 4.5 s, 4 s, 3.5 s, 3 s, 2.5 s, 2 s, 1.5 s, 1 s, 0.5 s, 0.25 s.
   In preferred embodiments, the sample is heated to the required temperature for around 1 second, for example between 0.5 seconds and 1.5 seconds.

In a particularly preferred embodiment, for example where the target nucleic acid is viral RNA, the sample is heated to between around 74° C. and 84° C., for example for around 1 second for example between 0.5 seconds and 1.5 seconds: or is heated to 95° C. for around 1 second, for example between 0.5 seconds and 1.5 seconds.

An additional issue is the thermal stability of the released RNA itself, RNA degrades in the presence (Non-Enzymatic RNA Hydrolysis Promoted by the Combined Catalytic Activity of Buffers and Magnesium Ions) of divalent cations in a basic solution. As a result, if the process described here is used with a standard Taq polymerase buffer based on Tris and 4M MgCl2 the released RNA is rapidly degraded and the target pathogen RNA is rendered non-amplifiable. To overcome this the applicants have developed buffers that are substantially pH neutral at the capsid Tm temperature and that become neutral above those temperatures, further the system buffers divalent cations (Free-metal ion depletion by “Good's” buffers, R Nakon, CR Krishnamoorthy, Science 19 Aug. 1983: Vol. 221, Issue 4612, pp. 749-750) such that the free concentrations are minimised, based on bicine and tricine—a typical formulation might be 50 mM bicine/tricine, 3.4 mM MgCl₂, 115 mM potassium acetate and adjusted to pH 8.2 at 25° C., this buffer will be essentially neutral at the Tm (capsid denaturation point) and as such there will be no excess hydroxyl ions to attack the RNA and yet the pH will be at the physiological requirement for the enzyme at the reverse transcription/amplification step, pH 7.4-7.6.

In some embodiments, the reagent is advantageously at a neutral pH, e.g. pH 6.5-7.5 at a temperature of between around 70° C.-100° C., for example between 72° C. and 98° C., 74° C. and 96° C., 76° C. and 94° C., 78° C. and 92° C., 80° C. and 90° C., 82° C. and 88° C., or between 84° C. and 86° C.: for example wherein the reagent is at pH 7 at a temperature of 95° C.

In some embodiments, the reagent also comprises one or more divalent cations. Polymerases typically require metal ions for activity and each enzyme may require a different cofactor. For example, DNA dependent DNA polymerases typically require Mg cations, whereas the RNA dependent DNA polymerase activity of some polymerases require Mn cations. Accordingly, in some embodiments the reagent comprises one or more divalent cations. The skilled person is well equipped to determine which co factors are required for which polymerase is being used.

It will be appreciated that there may be advantages if some or all of the components of the reagent are supplied lyophilised. For example, since the present invention is particularly suited for field based use in hot regions with poor access to refrigeration, supplying the components lyophilised reduces degradation or the components. Accordingly, in some embodiments the vessel may comprise lyophilised components, such as polymerases. The lyophilised components may also be supplied in a bulk volume. In the same or other embodiments, the lyophilised components may be resuspended in a “resuspension” reagent which comprises those components that cannot be supplied lyophilised. In a preferred embodiment, the lyophilised components are resuspended in the resuspension buffer and are placed in the vessel (if the lyophilised components are not supplied already in the vessel), prior to the sample being added to the vessel. The lid is then sealed shut and does not require opening again. The sample and vessel can then be safely destroyed once the amplification and detection have been performed.

Ultimately, following resuspension of any lyophilised components in the resuspension reagent, all components necessary for the amplification reaction to later take place are present, including the polymerase. The sample is then added prior to the sample being heated to at least 70° C. For example the resuspension buffer may consist of the glycerol, the Tween 20 and water and some salts such as magnesium chloride, to this resuspension buffer may be added an agent to prevent microbial growth such as sodium azide, though other antimicrobial agents are known in the art.

As discussed, the virus or microbe may comprise target nucleic acid that is DNA or RNA.

The skilled person will understand that to amplify DNA, for example with a PCR reaction, a single polymerase activity is required, i.e. DNA dependent DNA polymerase. The skilled person understands that there are many DNA dependent DNA polymerases available commercially, for example Taq polymerase, Vent polymerase, KOD, Tli

The skilled person also understands that in instances where the template target nucleic acid is RNA, to amplify the RNA it first has to be converted to cDNA using an RNA dependent RNA polymerase. Known RNA dependent polymerases include Bioneer Rocketscript, superscript, AMV, MMULV, FIV amongst others.

Reverse transcription by an RNA dependent DNA polymerase is followed by amplification of the resultant cDNA by a DNA dependent DNA polymerase. Accordingly, in some instances the reagent may comprise more than one polymerase, and can for example comprise an RNA dependent DNA polymerase and a DNA dependent DNA polymerase. As described above, a thermotolerant polymerase is required to survive the high temperatures associated with preparing the sample and the downstream amplification process.

However, preferably, where the template target nucleic acid is RNA, a single polymerase is present in the reagent that is capable of performing both the RNA dependent DNA polymerase function and the DNA dependent DNA polymerase function.

Accordingly, in one embodiment the reagent comprises at least two different polymerase enzymes as described above. Other combinations of enzymes are also contemplated, for example the reagent may comprise a polymerase with RNA dependent DNA polymerase activity and DNA dependent DNA polymerase activity, but the reagent may also comprise a further polymerase that has DNA dependent DNA polymerase activity which is considered may increase the fluorescence yield obtained during/following amplification. These may be included at a ratio of, for example 1:1, 2:1, 3:1 in favour of either polymerase. Suitable DNA polymerases include KOD, Tli, hemoTaq, Kapa blood, Phusion, TTH.

In the same or another embodiment the polymerase has RNA dependent DNA polymerase activity and does not have DNA dependent DNA polymerase activity, for example the polymerase is Bioneer Rocketscript.

The polymerase should be a polymerase wherein the RNA dependent DNA polymerase activity and/or the DNA dependent DNA polymerase activity can survive a brief hold at the temperature that the sample is heated to, for example at the temperature which renders the nucleic acid, for example viral nucleic acid, accessible to the amplification components.

In the same or another embodiment the polymerase has DNA dependent DNA polymerase activity and does not have an RNA dependent DNA polymerase activity.

In yet a further embodiment, the reagent comprises a polymerase with RNA dependent DNA polymerase activity but no DNA dependent DNA polymerase activity and also comprises a separate polymerase with DNA dependent DNA polymerase activity but no RNA dependent DNA polymerase activity.

In a preferred embodiment the polymerase has DNA dependent DNA polymerase activity; RNA dependent polymerase activity; or has both RNA dependent DNA polymerase activity and DNA dependent DNA polymerase activity, optionally wherein the polymerase is selected from the group consisting of:
a) TTH polymerase (Promega) [SEQ ID NO: 3]
b) Hawk Z05 (Roche), [SEQ ID NO: 4]
c) polymerases described in WO 2014/023318
d) a polymerase with at least 90%, 91%, 92%, 93%, 94%, 95%, 96%, 97%, 98%, 99% or 100% sequence identity to SEQ ID NO: 1, 2, 3, 4, optionally wherein the polymerase comprises 1, 2, 3, 4, 5, 6, 7 or 8 of the following mutations: S515R, I638F, M747K, L322M, L459M, S739G, E773G, and L789F with regard to SEQ ID NO: 1, 2, 3 or 4;
e) a polymerase with at least 90%, 91%, 92%, 93%, 94%, 95%, 96%, 97%, 98%, 99% or 100% sequence identity to M1/M747K enzyme [SEQ ID NO: 2];

A preferred enzyme has at least 90%, 91%, 92%, 93%, 94%, 95%, 96%, 97%, 98%, 99% or 100% sequence identity to SEQ ID NO: 1, 2, 3, 4, optionally wherein the polymerase comprises 1, 2, 3, 4, 5, 6, 7 or 8 of the following mutations: S515R, I638F, M747K, L322M, L459M, S739G, E773G, and L789F with regard to SEQ ID NO: 1, 2, 3 or 4; or is a polymerase with at least 90%, 91%, 92%, 93%, 94%, 95%, 96%, 97%, 98%, 99% or 100% sequence identity to M1/M747K enzyme [SEQ ID NO: 2]; since certain of these enzymes are considered to be more tolerant of the presence of blood during amplification than Hawkz05 and is better at reverse transcription than TTH. Amplification data produced by such an enzyme is provided in the examples.

It is also preferable if the polymerase(s) are either naturally resistant to, or have been engineered to be resistant to, inhibitors found in some samples such as blood. For example, the enzyme TTH is considered to be naturally resistant to inhibitors present in blood (Scientific Reports volume 8, Article number: 3410 (2018)). Certain enzymes with at least 90%, 91%, 92%, 93%, 94%, 95%, 96%, 97%, 98%, 99% or 100% sequence identity to SEQ ID NO: 1, 2, 3, 4, optionally wherein the polymerase comprises 1, 2, 3, 4, 5, 6, 7 or 8 of the following mutations: S515R, I638F, M747K, L322M, L459M, S739G, E773G, and L789F with regard to SEQ ID NO: 1, 2, 3 or 4; or enzymes with at least 90%, 91%, 92%, 93%, 94%, 95%, 96%, 97%, 98%, 99% or 100% sequence identity to M1/M747K enzyme [SEQ ID NO: 2]; are considered resistant to the inhibitors found in blood.

SEQ ID NO: 1 refers to the amino acid sequence of Thermus aquaticus
polymerase:
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESPKALEEAPWPPPEGAFVGFVLSRKEPMWADLLALAAAR
GGRVHRAPEPYKALRDLKEARGLLAKDLSVLALREGLGLPPGDDPMLLAYLLDP
SNTTPEGVARRYGGEWTEEAGERAALSERLFANLWGRLEGEERLLWLYREVERP
LSAVLAHMEATGVRLDVAYLRALSLEVAEEIARLEAEVFRLAGHPFNLNSRDQLE
RVLFDELGLPAIGKTEKTGKRSTSAAVLEALREAHPIVEKILQYRELTKLKSTYIDP
LPDLIHPRTGRLHTRFNQTATATGRLSSSDPNLQNIPVRTPLGQRIRRAFIAEEGWL
LVALDYSQIELRVLAHLSGDENLIRVFQEGRDIHTETASWMFGVPREAVDPLMRR
AAKTINFGVLYGMSAHRLSQELAIPYEEAQAFIERYFQSFPKVRAWIEKTLEEGRR
RGYVETLFGRRRYVPDLEARVKSVREAAERMAFNMPVQGTAADLMKLAMVKLF
PRLEEMGARMLLQVHDELVLEAPKERAEAVARLAKEVMEGVYPLAVPLEVEVGI
GEDWLSAKE
SEQ I NO: 2 refers to the M1 polymerase with a M747K substitution, as
described in WO 2014/023318:
MRGMLPLFEPKGRVLLVDGHHLAYRTFHALKGLTTSRGEPVQAVYGFAKSLLK
ALKEDGDAVIVVFDAKAPSFRHEAYGGYKAGRAPTPEDFPRQLALIKELVDLLGL
ARLEVPGYEADDVLASLAKKAEKEGYEVRILTADKDLYQLLSDRIHVLHPEGYLI
TPAWLWEKYGLRPDQWADYRALTGDESDNLPGVKGIGEKTARKLLEEWGSLEA
LLKNLDRLKPAIREKILAHMDDLKLSWDLAKVRTDLPLEVDFAKRREPDRERLR
AFLERLEFGSLLHEFGLLESPKALEEAPWPPPEGAFVGFVLSRKEPMWADLMALA
AARGGRVHRAPEPYKALRDLKEARGLLAKDLSVLALREGLGLPPGDDPMLLAY
LLDPSNTTPEGVARRYGGEWTEEAGERAALSERLFANLWGRLEGEERLLWLYRE
VERPLSAVLAHMEATGVRLDVAYLRAMSLEVAEEIARLEAEVFRLAGHPFNLNS
RDQLERVLFDELGLPAIGKTEKTGKRSTRAAVLEALREAHPIVEKILQYRELTKLK
STYIDPLPDLIHPRTGRLHTRFNQTATATGRLSSSDPNLQNIPVRTPLGQRIRRAFIA
EEGWLLVALDYSQIELRVLAHLSGDENLIRVFQEGRDFHTETASWMFGVPREAV
DPLMRRAAKTINFGVLYGMSAHRLSQELAIPYEEAQAFIERYFQSFPKVRAWIEK
TLEEGRRRGYVETLFGRRRYVPDLEARVKGVREAAERMAFNKPVQGTAADLMK
LAMVKLFPRLGEMGARMLLQVHDELVLEAPKERAEAVARLAKEVMEGVYPLA
VPLEVEVGIGEDWLSAKE
SEQ ID NO: 3 refers to the TTH polymerase sequence:
MEAMLPLFEPKGRVLLVDGHHLAYRTFFALKGLTTSRGEPVQAVYGFAKSLLKA
LKEDGYKAVFVVFDAKAPSFRHEAYEAYKAGRAPTPEDFPRQLALIKELVDLLG
FTRLEVPGYEADDVLATLAKKAEKEGYEVRILTADRDLYQLVSDRVAVLHPEGH
LITPEWLWEKYGLRPEQWVDFRALVGDPSDNLPGVKGIGEKTALKLLKEWGSLE
NLLKNLDRVKPENVREKIKAHLEDLRLSLELSRVRTDLPLEVDLAQGREPDREGL
RAFLERLEFGSLLHEFGLLEAPAPLEEAPWPPPEGAFVGFVLSRPEPMWAELKAL
AACRDGRVHRAADPLAGLKDLKEVRGLLAKDLAVLASREGLDLVPGDDPMLLA
YLLDPSNTTPEGVARRYGGEWTEDAAHRALLSERLHRNLLKRLEGEEKLLWLYH
EVEKPLSRVLAHMEATGVRLDVAYLQALSLELAEEIRRLEEEVFRLAGHPFNLNS
RDQLERVLFDELRLPALGKTQKTGKRSTSAAVLEALREAHPIVEKILQHRELTKL
KNTYVDPLPSLVHPRTGRLHTRFNQTATATGRLSSSDPNLQNIPVRTPLGQRIRRA
FVAEAGWALVALDYSQIELRVLAHLSGDENLIRVFQEGKDIHTQTASWMFGVPP
EAVDPLMRRAAKTVNFGVLYGMSAHRLSQELAIPYEEAVAFIERYFQSFPKVRA
WIEKTLEEGRKRGYVETLFGRRRYVPDLNARVKSVREAAERMAFNMPVQGTAA
DLMKLAMVKLFPRLREMGARMLLQVHDELLLEAPQARAEEVAALAKEAMEKA
YPLAVPLEVEVGMGEDWLSAKG
SEQ ID NO: 4 refers to the HawkZ05 polymerase sequence:
MKAMLPLFEPKGRVLLVDGHHLAYRTFFALKGLTTSRGEPVQAVYGFAKSLLKA
LKEDGY
KAVFVVFDAKAPSFRHEAYEAYKAGRAPTPEDFPRQLALIKELVDLLGFTRLEVP
GFEAD
DVLATLAKKAEREGYEVRILTADRDLYQLVSDRVAVLHPEGHLITPEWLWEKYG
LKPEQW
VDFRALVGDPSDNLPGVKGIGEKTALKLLKEWGSLENILKNLDRVKPESVRERIK
AHLED
LKLSLELSRVRSDLPLEVDFARRREPDREGLRAFLERLEFGSLLHEFGLLEAPAPL
EEAP
WPPPEGAFVGFVLSRPEPMWAELKALAACKEGRVHRAKDPLAGLKDLKEVRGL
LAKDLAV
LALREGLDLAPSDDPMLLAYLLDPSNTTPEGVARRYGGEWTEDAAHRALLAERL
QQNLLE
RLKGEEKLLWLYQEVEKPLSRVLAHMEATGVRLDVAYLKALSLELAEEIRRLEE
EVFRLA
GHPFNLNSRDQLERVLFDELRLPALGKTQKTGKRSTSAAVLEALREAHPIVEKIL
QHREL
TKLKNTYVDPLPGLVHPRTGRLHTRFNQTATATGRLSSSDPNLQNIPIRTPLGQRI
RRAF
VAEAGWALVALDYSQIELRVLAHLSGDENLIRVFQEGKDIHTQTASWMFGVSPE
AVDPLM
RRAAKTVNFGVLYGMSAHRLSQELAIPYEEAVAFIERYFQSFPKVRAWIEKTLEE
GRKRG
YVETLFGRRRYVPDLNARVKSVREAAERMAFNMPVQGTAADLMKLAMVKLFP
HLREMGAR
MLLQVHDELLLEAPQARAEEVAALAKEAMEKAYPLAVPLEVEVGIGEDWLSAK
G

It will be appreciated that in instances where the method of preparing the sample, and the subsequent amplification and detection of the product are to be “closed-tube”, i.e. a situation where once the sample and materials necessary for amplification and detection are placed within the vessel the vessel is closed and is not opened again, and wherein polymerases are present such that both RNA dependent DNA polymerase and DNA dependent DNA polymerase activity are present (either by virtue of at least two different polymerases being present in the vessel, one with RNA dependent DNA polymerase activity and another with DNA dependent DNA polymerase activity; or where a single polymerase is present that has both activities; or where a single enzyme is present that has both activities and at least one further enzyme is present that has DNA dependent DNA polymerase activity or RNA dependent DNA polymerase activity) it is preferred that the RNA dependent DNA polymerase activity does not require a cofactor that is an inhibitor of the DNA dependent DNA polymerase activity, and/or the DNA dependent DNA polymerase activity does not require a cofactor that is an inhibitor of the RNA dependent DNA polymerase activity.

In a preferred embodiment, the RNA dependent DNA polymerase activity and the DNA dependent DNA polymerase activity both require magnesium cations as a cofactor. It will be clear to the skilled person that where the target nucleic acid is RNA, for example where the virus is an RNA virus, it is preferred if the polymerase has RNA dependent DNA polymerase activity, or for example has both RNA dependent DNA polymerase activity and DNA dependent DNA polymerase activity.

As discussed above, the sample may be any sample type, but preferably is a crude sample. The sample may be of any volume and may be of any relative volume with respect to the combined volume of the sample and reagent. However, the present method is considered suitable for use with high relative volume samples, for example in one embodiment the sample makes us at least 5% of total volume of the sample and reagent, for example at least 6%, 7%, 8%, 9%, 10%, 1%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% or 35% or greater: optionally comprises 13% of total volume of the sample and reagent.

As described in the examples, where the sample is blood or serum, the preferred concentration is around 12-16% blood or serum. Without wishing to be bound by any theory, it is thought that molecular crowding at these concentrations increases the efficiency of the amplification reaction.

In this way it is possible to use a sufficiently large sample, for example crude sample for example whole blood, but still perform amplification and detection in a small volume. For example, in some embodiments, the volume of sample, for example crude sample for example whole blood is less than 100 μl, for example less than 90 μl, 80 μl, 70 μl, 60 μl, 50 μl, 40 μl, 30 μl, 20 μl, 15 μl, 12 μl, 10 μl, 5 μl, 4 μl, 3 μl, 2 μl, 1 μl, 0.5 μl. The use of small volumes of sample reduces the risk of exposure to the virus or microbe. For example the sample volume may be between 0.5 μl and 100 μl, for example between 1 μl and 90 μl, for example between 2 μl and 80 μl, 3 μl and 70 μl, 4 μl and 60-μl, 5 μl and 50 μl, 10 μl and 40 μl, 15 μl and 30 μl or 20 μl. In some preferred embodiments the crude sample is 5 μl, 10 μl, 12 μl, 15 μl or 20 μl.

The total volume of the sample and reagent in the vessel may be between 10 μl and 500 μl, 20-μl and 450-μl, 30 μl and 400 μl, 40 μl and 350 μl, 50 μl and 300 μl, 60 μl and 250 μl, 70 μl and 200 μl, 80 μl and 150 μl, 90 μl and 140 μl, 100 μl and 130 μl, 110 μl and 120 μl. For example the total volume may be less than 500 μl, 400 μl, 300 μl, 200 μl, 100 μl, 90 μl, 80 μl, 70 μl, 60 μl, 50 μl, 40 μl, 30 μl, 20 μl or 10 μl. In some preferred embodiments the total reaction volume is 50 μl, 60 μl, 70 μl, 80 μl, 90 μl, 62 μl, 72 μl, 82 μl, 92 μl, 102 μl, 75 μl, 85 μl, 95 μl, 105 μl, 110 μl.

Accordingly, in some embodiments:
a) the volume of the sample in the vessel is
i) less than 100 μl, for example less than 90 μl, 80 μl, 70 μl, 60 μl, 50 μl, 40 μl, 30 μl, 20 μl, 15 μl, 10 μl, 5 μl, 4 μl, 3 μl, 2 μl, 1 μl, 0.5 μl; and/or
ii) between 0.5 μl and 100 μl, for example between 1 μl and 90 μl, for example between 2 μl and 80 μl, 3 μl and 70 μl, 4 μl and 60 μl, 5 μl and 50 μl, 10 μl and 40 μl, 15 μl and 30 μl or 20 μl; and/or
b) the total volume of the sample and reagent in the vessel may be between
i) 10 μl and 500 μl, 20 μl and 450 μl, 30 μl and 400 μl, 40 μl and 350 μl, 50 μl and 300 μl, 60 μl and 250 μl, 70 v and 200 μl, 80 v and 150 μl, 90 μl and 140 μl, 100 μl and 130 μl, 110 μl and 120 μl; and/or
ii) may be less than 500 μl, 400 μl, 300 μl, 200 μl, 100 μl, 90 μl, 80 μl, 70 μl, 60 μl, 50 μl, 40 μl, 30 μl, 20 μl or 10 μl.

In some preferred embodiments, the volume of the sample is 10 μl and the total volume of the sample and reagent in the vessel is 60 μl, 70 μl, 80 μl, or 90 μl, or the volume of the sample is 12 μl and the total volume of sample and reagent in the vessel is 62 μl, 72 μl, 82 μl, 92 μl, 102 μl, or the volume of the sample is 15 μl and the total volume is 75 μl, 85 μl, 95 μl or 105 μl.

As described above, in some embodiments the method is field based and is suitable for use in the field. By field use we include the meaning of use in a non-standard laboratory setting. In one embodiment by field use we mean for use in an environment where electricity is limited, or available only by battery. In the same or a different embodiment, by field use we include the meaning of situations wherein the clinical practitioner, laboratory scientist, or other person who handles the vessel is not equipped with suitable safety equipment for the nature of the substance for which the vessel is to be used. For example, in some embodiments the vessel is to be used to detect highly pathogenic viruses and bacteria and will typically be used to house a sample from a subject, or an environmental sample and in certain situations, such as in field use, the person handling the vessel may be equipped with only very basic safety equipment. We also include the meaning of the method is low cost, simple and biosafe, requiring minimal exposure to the sample and once the sample has been added to the vessel the vessel is not opening again. We also include the meaning that the method can be performed using minimal energy, such as electricity, and does not require the sample to be frozen, centrifuged, and does not require amplification or reagent components that necessarily require refrigeration or freezing.

In a preferred embodiment, the sample is taken directly from the subject and placed directly into the vessel, along with the reagent.

In some embodiments, particularly where the sample comprises blood or comprises whole blood, the reagent comprises an agent that aids in blood coagulation. As discussed above, the presence of blood both interferes with fluorescent detection of amplified products and can inhibit amplification itself due to the presence of PCR inhibitors. The inventors have found that if the blood is coagulated it will relatively rapidly sink to the bottom of the vessel, for example during the time course of the amplification reaction. The coagulated blood, though available for providing viral or microbial nucleic acid to the vessel, is less able to leach out amplification inhibitors.

As will be apparent from the above, the present invention provides a method for preparing a sample for the direct amplification of microbial or viral nucleic acid present.

Accordingly, it follows that the invention also provides a method of amplifying microbial or viral nucleic acid present in a sample obtained from a subject wherein the sample is prepared according to the method of the invention. Preferences given for features of the method of preparing a sample apply to throughout the specification, for example apply to the method of amplification. For example, the preferences for the sample type, volumes, reagent components, temperatures, polymerases etc given above apply to the method of amplification.

In one embodiment the amplification is performed using PCR or a q-PCR. In some embodiments the amplification involves a reverse transcription step before PCR, for example a reverse transcription PCR (RT-PCR), for example real-time reverse transcription PCR (RT-qPCR).

The inventors have surprisingly found that it is possible and beneficial to perform more than one cycle of reverse transcriptase in situations where the target nucleic acid is RNA, for example viral RNA, and particularly beneficial where the abundance of the RNA is low. For example, reverse transcription is typically only 10-20% efficient (Miranda J A: Steward G F, Variables influencing the efficiency and interpretation of reverse transcription quantitative PCR (RT-qPCR): An empirical study using Bacteriophage MS2. J Virol Methods. 2017; 241: 1-10) and so in situations where the copy number of the RNA is expected to be less than 100 copies per reaction, multiple rounds of reverse transcription is considered to be particularly beneficial.

In this way the reverse transcription of the RNA to cDNA is repeated, and requires heating the sample to a high enough temperature to separate the strands of the resulting cDNA hybrid, for example heating to 95 C. The skilled person will appreciate that this embodiment requires a polymerase that can withstand heating to 95 C.

Whilst other methods of apparent cyclical RT have been reported (e.g. Bioneer WO2008115002), those methods never achieve the denaturation temperature of cDNA. It is well established the melting point of a DNA/RNA hybrid is higher than that of the corresponding DNA, as a result it necessary to heat any reaction to in excess of 94 C to reliably denature the resulting hybrids if the amplicon length is in excess of 80 bp. This means that the maximum described temperature in that publication is incapable of denaturing the hybrids and as such could never make more cDNA molecules than the number of RNA molecules present. BioRad described a method of performing at least two rounds of reverse transcription in WO2014138688, however this describes a compartmentalised reaction strategy where each sample is broken down into multiple nanolitre reactions, each of which in theory being a single target per reactions. This is a major differentiation as it requires extracted nucleic acids, there are not enough pathogens per nanolitre in a crude sample for this method to work and it specifically claims compartmentalised reactions. In essence this is a method of ensuring that the single target in each partition is successfully transcribed as opposed to a method designed to generate more cDNA molecules than the amount of RNA originally present as described here. This pre-amplification is key to maximising sensitivity when very low number of targets are expected to be present in the reaction.

The method may involve any number of cycles of reverse transcription. By a cycle of reverse transcription we include the meaning of allowing the RNA dependent DNA polymerase activity of a suitable enzyme to reverse transcribe the RNA into a strand of DNA. A second cycle would require heating the sample to a threshold temperature at which the RNA:DNA hybrid disassociates, allowing the polymerase to access the RNA. The temperature is lowered to the extension temperature and a second strand of DNA is generated from the same template RNA molecule.

In some embodiments the amplification involves more than one reverse transcription step, i.e. involves repetitive reverse transcription, for example at least 2, 3, 4, 5, 6, 7, 8, 9, 10, 11, 12, 13, 14, 15, 16, 17, 18, 19, 20 or more than 20 cycles of reverse transcription before PCR.

In some embodiments the reverse primer that drives the reverse transcription is the same as the reverse primer that drives the PCR.

The applicants in this case have found a further improvement, that of manipulating the melting point of the primer driving the reverse transcription such that it is possible to separate RT from PCR or indeed to maximise the performance of both. If the Tm, the point at which 50% of the primer is bound to the intended target, of the primer driving reverse transcription is separated from its Tm to DNA the applicants have shown that it is possible to bias the reaction to reverse transcription only, i.e the reaction can be run at a temperature where only cDNA is formed and the PCR reaction cannot be initiated as the reverse transcription is being performed at too high a temperature for the primer to bind to any DNA in the reaction. One such modification is the incorporation of locked nucleic acids into the reverse primer sequence, which as (Locked nucleic acids in PCR primers increase sensitivity and performance. Genomics, ISSN: 1089-8646, Vol: 91, Issue: 3, Page: 301-5, 2008) modified RNA molecule disproportionately increases the Tm to RNA templates and as such biases the reaction to the generation of cDNA. For example the sequence GAGTAGGAG{T}{T}{T}GTGAAAGTGTC (SEQ ID NO:21) (where the brackets represent LNA modifications), has a Tm to DNA of 64 C but of 78 C to RNA. Therefore it is possible to reverse transcribe at for example 75C and yet generate no PCR product, therefore the repetitive reverse transcription is characterised by a reverse primer that has a Tm that is minimally 9 C hotter than that to the corresponding DNA target and running the reverse transcription at 6 C or greater than the TM (to DNA). At this juncture less than 1% of the primer may bind to the DNA target and as such largely reverse transcription takes place and as such it represents a pre-amplification step, which is vital in this approach where infected blood will only contain 3 targets per microlitre of blood when the patient is infected with 3,000 virions per ml. Standard Rt-QPCR requires tens of copies to be present before a reaction will initiate and as such this pre-step represents a necessary boost to diagnostic sensitivity

The primers driving the repetitive reverse transcription and the PCR can be designed so as to either a) separate reverse transcription from PCR, based on temperature; or b) allow concurrent reverse transcription and PCR.

In some embodiments the melting point of the reverse primer driving the reverse transcription is different to the melting point of the forward primer driving PCR so that depending on the temperature of the reaction a) reverse transcription occurs, b) PCR occurs, or c) both reverse transcription and PCR occurs simultaneously. If the Tm to DNA of the forward primer is separated from that of the reverse to the RNA target by more than 6 C then it is possible to perform efficient reverse transcription while preventing PCR taking place.

In a preferred embodiment, the difference in the melting points of the primer is around 5-6 C.

The skilled person will understand that the Tm of the primers to RNA or DNA is driven largely by the GC content of the sequences.

In some embodiments the temperature of the reverse transcription reaction exceeds the melting temperature of the DNA/RNA hybrid.

In some embodiments one or more primers comprise one or more LNA, ZNA and/or BNA modifications, for example in some embodiments the reverse primer comprises one or more LNA, ZNA and/or BNA modifications. One or more primers may also, or instead, comprise one or more LNA, ZNA and/or BNA modifications.

The skilled person will appreciate that amplification such as PCR or RT-PCR or RT-qPCR can be used to generate amplicons of a wide range of lengths for example from 20 bp to 5,000 bp. However, for the present purpose, i.e. disease diagnosis, the shorter amplicons are preferred since a shorter amplification reaction is required meaning that diagnostic results can be obtained more quickly. Accordingly in some embodiments the amplification results in an amplicon with a length of between 40 bp and 500 bp, for example between 50 bp and 450 bp, for example between 60 bp and 400 bp, for example between 70 bp and 350 bp, for example between 80 bp and 300 bp, for example between 90 bp and 250 bp, for example between 100 bp and 200 bp, for example around 150 bp. Preferably the size of the resultant amplicon is 60 bp to 100 bp.

As discussed above, there are benefits associated with performing the amplification reaction in the same vessel that the sample has been added to and in which the sample has been prepared, for example biosecurity reasons. Accordingly in one embodiment the amplification is performed in the same vessel as that in which the sample has been prepared according to the method of preparing the sample according to the invention.

In another embodiment once the sample and reagent are in the vessel, the vessel is sealed and is not opened again throughout the amplification reaction.

In the same or different embodiment, once the sample and reagent are in the vessel no part of the sample or reagent is removed from the vessel:
a) prior to said heating; and/or
b) following said heating;
c) prior to RT; and/or
d) prior to PCR; and/or
e) during RT; and/or
f) during PCR; and/or
g) following RT; and/or
h) following PCR.

In some embodiments the method is a closed-tubed method of amplifying the nucleic acid. The skilled person will understand what is meant by a closed-tubed method and typically requires that once the sample has been added to the vessel, along with any necessary components for the method of preparing, or downstream amplification and detection steps, the vessel is closed, for example by closing a cap or sealing a lid onto the vessel, and the cap or lid is not opened again. For example in some embodiments of the closed tube method no further material is added or removed from the vessel, for example the nucleic acid is not extracted or in any way purified.

The skilled person will understand that amplification, e.g. PCR is performed using at least a forward and reverse primer. In some instance, such as qPCR or RT-qPCR, the primers may be labelled with suitable fluorophores or other dyes that allow detection of the amplicon. The reaction may also use probes, such a fluorescently labelled probes. Where the sample is blood and the blood sinks out of the light path, or for samples other than blood, suitable fluorophores span the collection wavelengths from FAM at 500 nm to alexa fluor 680 at 750 nm and encompass any fluorophores known in the art, for example TET, HEX, Cy5. Without sinking (or centrifugation), blood samples require far red dyes include Cy5, alexa fluor 657, 680, 594, pulsar 650, quasar 670, CY5.5, quasar 705 amongst others emitting between 630 and 750 nm.

Since in preferred embodiments the sample is not disturbed once it has been added to the vessel, in some embodiments the sample and reagent is not manipulated between the reverse transcriptase step and the PCR step. [One step RT-PCR]

The skilled person will appreciate that as well as providing a method of preparing a sample for amplification and providing a method of amplification, the invention also provides a method of detecting the presence of a microbe or viral particle in a sample obtained from a subject wherein the sample has been prepared according to the invention and/or wherein the nucleic acid from the microbe or viral particle is amplified according to the invention, followed by detection of the amplified nucleic acid.

As described above, in some embodiments the amplification results in a fluorescent signal that corresponds to the quantity of amplicon. For example fluorescently labeled primers may become incorporated into the amplicon, or fluorescently labelled probes can be used to quantify the amount of amplicon made, or the presence of amplicon. The skilled person is aware of quantitative PCR (qPCR/RT-qPCR) and the available options for performing the reaction and detecting the amplicon.

In some embodiments the amplicon is detected with a spectrophotometer, for example when fluorescently labeled dyes/primers have been used in the reaction.

The skilled person will appreciate that spectrophotometers can capture all emitted light, for example between 300-900 nm and then filters can be used that allow it to see in particular ranges, for example in two windows of 510-580 nm and 655-750 nm. This means that you can use any dyes that fall in those wavelengths and not need to calibrate the samples or calculate for spectral overlap or any of the other issues with normal optical systems.

Accordingly in one embodiment the amplicon is labelled during amplification with fluorescently labelled primer(s) or probe(s) and the resultant fluorescence is detected using a spectrophotometer that captures all light between 300-900 nm with two windows of 510-580 nm and 655-750 nm.

For non-pathogenic viruses or microbes, or pathogenic viruses or microbes that are not considered dangerous, the closed-tube embodiments of the present invention are considered to be less critical and so other means of detecting the amplicon, such as electrophoresis, can be used, and/or the amplicon may be sequenced for epidemiological study.

The invention also provides a reagent for use in any of the methods of the invention, for example for use in the preparation of a sample and/or for use in the method of amplification of the invention, and/or according for use in the method of detection according to the invention, wherein the reagent comprises a detergent, a solvent and one or more nucleic acid polymerases.

Preferences for features of this aspect of the invention are as defined elsewhere herein, for example the preferences for the solvent, detergent, polymerase, concentrations of each component, pH etc are as defined herein.

As discussed above, in some embodiments the reagent minimises RNA degradation.

Also as discussed above in some embodiments the reagent does not comprise Tris.

The reagent may comprise bicine, for example may comprise bicine at a concentration of between 20 mM and 70 mM Bicine, for example between 25 mM and 65 mM Bicine, for example between 30 mM and 60 mM Bicine, for example between 35 mM and 55 mM Bicine, for example between 40 mM and 50 mM Bicine, for example 50 mM Bicine.

In preferred embodiments the reagent buffers divalent cations.

In some embodiments the solvent is glycerol and/or the detergent is Tween, for example Tween 20. In particular embodiments the reagent comprises Tween, for example Tween20 at a concentration of up to 0.4% and/or comprises glycerol at a concentration of up to 11%, for example wherein the reagent comprises 11%, 10%, 9%, 8%, 7%, 6%, 5%, 4%, 3%, 2%, 1%, 0.5% or less glycerol.

As described above, the reagent comprises one or more polymerases. In some embodiments the reagent comprises at least two different polymerase enzymes.

For example in one embodiment the polymerase has DNA dependent DNA polymerase activity: RNA dependent polymerase activity: or has both RNA dependent DNA polymerase activity and DNA dependent DNA polymerase activity, optionally wherein the polymerase is selected from the group consisting of:
a) TTH polymerase (Promega) [SEQ ID NO: 3]
b) Hawk Z05 (Roche), [SEQ ID NO: 4]
c) polymerases described in WO 2014/023318
d) a polymerase with at least 90%, 91%, 92%, 93%, 94%, 95%, 96%, 97%, 98%, 99% or 100% sequence identity to SEQ ID NO: 1, 2, 3, 4, optionally wherein the polymerase comprises 1, 2, 3, 4, 5, 6, 7 or 8 of the following mutations: S515R, I638 F, M747K, L322M, L459M, S739G, E773G, and L789F with regard to SEQ ID NO: 1, 2, 3 or 4;
e) a polymerase with at least 90%, 91%, 92%, 93%, 94%, 95%, 96%, 97%, 98%, 99% or 100% sequence identity to M1/M747K enzyme [SEQ ID NO: 2]:

A preferred enzyme has at least 90%, 91%, 92%, 93%, 94%, 95%, 96%, 97%, 98%, 99% or 100% sequence identity to SEQ ID NO: 1, 2, 3, 4, optionally wherein the polymerase comprises 1, 2, 3, 4, 5, 6, 7 or 8 of the following mutations: S515R, I638F, M747K, L322M, L459M, S739G, E773G, and L789F with regard to SEQ ID NO: 1, 2, 3 or 4; or is a polymerase with at least 90%, 91%, 92%, 93%, 94%, 95%, 96%, 97%, 98%, 99% or 100% sequence identity to M1/M747K enzyme [SEQ ID NO: 2]; since certain of these enzymes are considered to be more tolerant of the presence of blood during amplification than Hawkz05 and is better at reverse transcription than TTH. Amplification data produced by such an enzyme is provided in the examples.

It is also preferable if the polymerase(s) are either naturally resistant to, or have been engineered to be resistant to, inhibitors found in some samples such as blood. For example, the enzyme TTH is considered to be naturally resistant to inhibitors present in blood. Certain enzymes with at least 90%, 91%, 92%, 93%, 94%, 95%, 96%, 97%, 98%, 99% or 100% sequence identity to SEQ ID NO: 1, 2, 3, 4, optionally wherein the polymerase comprises 1, 2, 3, 4, 5, 6, 7 or 8 of the following mutations: S515R, I638F, M747K, L322M, L459M, S739G, E773G, and L789F with regard to SEQ ID NO: 1, 2, 3 or 4; or enzymes with at least 90%, 91%, 92%, 93%, 94%, 95%, 96%, 97%, 98%, 99% or 100% sequence identity to M1/M747K enzyme [SEQ ID NO: 2]; are considered resistant to the inhibitors found in blood.

The reagent may also comprise a first polymerase with RNA dependent DNA polymerase activity and a second polymerase with DNA dependent DNA polymerase activity.

The reagent may also comprise a first polymerase with RNA dependent DNA polymerase activity and DNA dependent DNA polymerase activity and a second polymerase with DNA dependent DNA polymerase activity.

In either case, the reagent may comprise a ratio of first to second polymerase of between 1:1 to 5:1, for example may comprise a ratio of first to second polymerase of at least 1:1, 1:2, 1:3, 1:4, or at least 1:5.

The reagent may also comprise a ratio of second to first polymerase of between 1:1 to 5:1, for example may comprise a ratio of first to second polymerase of at least 1:1, 1:2, 1:3, 1:4, or at least 1:5.

For example the reagent may comprise a first polymerase with RNA dependent DNA polymerase activity and a second polymerase with DNA dependent DNA polymerase activity wherein the ratio of the first polymerase to the second polymerase is between 1:1 to 5:1, for example may comprise a ratio of first to second polymerase of at least 1:1, 1:2, 1:3, 1:4, or at least 1:5; or wherein the ratio of the second to first polymerase is between 1:1 to 5:1, for example may comprise a ratio of first to second polymerase of at least 1:1, 1:2, 1:3, 1:4, or at least 1:5.

In another example the reagent may comprise a first polymerase with RNA dependent DNA polymerase activity and DNA dependent DNA polymerase activity and a second polymerase with DNA dependent DNA polymerase activity wherein the ratio of the first polymerase to the second polymerase is between 1:1 to 5:1, for example may comprise a ratio of first to second polymerase of at least 1:1, 1:2, 1:3, 1:4, or at least 1:5; or wherein the ratio of the second to first polymerase is between 1:1 to 5:1, for example may comprise a ratio of first to second polymerase of at least 1:1, 1:2, 1:3, 1:4, or at least 1:5.

The reagent may comprise 1, 2, 3, 4, 5 or more different polymerase enzymes, with the same or different activities.

Further, in some embodiments the reagent comprises an agent that aids in blood coagulation. Preferences for the agent that aids in blood coagulation are as described elsewhere herein.

It will be appreciated that since in some embodiments the amplification reaction is a closed-tube biosafe reaction, the reagent ought to comprise all components necessary for amplification and subsequent detection, bar the sample itself. Accordingly in one embodiment the reagent comprises components necessary for PCR and/or RT-PCR and or RT-qPCR, for example may comprise:
one or more primers, for example one or more fluorophore labelled primers; and/or one or more fluorescent dyes:
   magnesium chloride; BSA; dNTP; arginine; random RNA e.g. yeast RNA; excipients needed to maintain enzyme activity whilst lyophilised.

Also as described above, some of the components of the reagent may be lyophilised which has advantages since refrigeration of lyophilised components is not typically required. Accordingly, in one embodiment one or more of the components of the reagent is in lyophilised form, optionally in lyophilised form in a vessel. In one embodiment one or more of the components of the reagent is in lyophilised form, optionally in lyophilised form in a vessel, optionally where one or more of the polymerase, BSA, primer(s), probe(s) or dNTPs are lyophilised, optionally are lyophilised together.

As described above the inventors have discovered that it is possible and beneficial to perform multiple rounds of reverse transcription. Multiple rounds of reverse transcription increase the sensitivity of the target nucleic acid amplification and detection and it made possible largely by the use of the reagent of the invention which protects the sample, particularly any RNA in the sample, from degradation. The invention therefore also provides a method of performing RT-PCR wherein the method comprises more than one reverse transcription step, for example 2, 3, 4, 5, 6, 7, 8, 9, 10, 11, 12, 13, 14, 15, 16, 17, 18, 19, 20 or more than 20 steps of reverse transcription prior to or during PCR. This method is considered to be independent of the other methods of the invention, but can be used in conjunction with the other methods. Preferences for the reverse transcriptase and repetitive reverse transcription are as described elsewhere here.

As described above, in the method of repetitive RT, in some embodiments the reverse primer that drives the reverse transcription is the same as the reverse primer that drives the PCR.

Further, and also as described above, in some embodiments the melting point of the reverse primer driving the reverse transcription is different to the melting point of the forward or reverse primers driving PCR so that depending on the temperature of the reaction a) reverse transcription occurs, b) PCR occurs, or c) both reverse transcription and PCR occurs simultaneously. For example, the difference in the melting points of the primer may be around 5 C or greater and the reverse transcription occurs 6 C or greater than the TM to DNA, such that only RT can take place.

The primers may comprise one or more LNA, ZNA and/or BNZ modifications, optionally wherein the reverse primer comprises one or more LNA, ZNA and/or BNZ modifications.

In some embodiments the temperature of the reverse transcription reaction exceeds the melting temperature of the DNA/RNA hybrid.

The repetitive RT can be used to amplify any size of amplicon. However in preferred embodiments the amplification results in an amplicon with a length of between 40 bp and 500 bp, optionally between 50 bp and 450 bp, optionally between 60 bp and 400 bp, optionally between 70 bp and 350 bp, optionally between 80 bp and 300 bp, optionally between 90 bp and 250 bp, optionally between 100 bp and 200 bp, optionally around 150 bp. The amplicons will preferably be in the range of 60-100 bp.

The invention also provides a method to determine the temperature at which viral nucleic acid becomes available for amplification wherein
a) the method comprises preparing multiple samples according to the method of the invention wherein the multiple samples are each individually heated to one of a range of different temperatures before PCR or RT-PCR, for example wherein individual samples are prepared and heated to one of 71, 72, 73, 74, 75, 76, 77, 78, 79, 80, 81, 82, 83, 84, 85, 86, 87, 88, 89, 90° C., 91° C., 92° C., 93° C., 94° C., 95° C. optionally heated to 76, 77, 78, 79, 80, 81° C.: followed by
b) amplification of the viral nucleic acid, for example amplification according to the method of the invention: followed by
c) detection of the amplicon, optionally detection of the amplicon according to the invention.

It will be clear that the methods described here in can be used to determine the presence or absence of a virus or microbe in a sample and therefore can be used in methods of diagnosis. Accordingly the invention provides a method for the diagnosis of the presence or absence of a microbial or viral infection wherein a sample obtained from a subject is prepared according to the invention, followed by:
a) amplification of the viral nucleic acid, for example amplification according the method of the invention: followed by
b) detection of the amplicon, for example detection of the amplicon according to the invention,
wherein the detection of the presence of the amplicon indicates that the subject has the microbial or viral infection.

It will also be clear to the skilled person that various aspects and embodiments of the present invention lend themselves to being provided as a kit. For example, the invention provides a kit comprising a detergent, a solvent and one or more nucleic acid polymerases. Preference for the detergent, solvent and polymerase(s) are as defined herein. In some embodiments the kit also comprises a reaction vessel, for example a vessel that comprises 35 an irreversible lock so that once the sample has been added and the vessel is sealed, the vessel cannot be opened again. Such vessels are described in PCT/GB2019/051156.

Various components of the kit may be in liquid form or lyophilised form. Preferences for lyophilised components are described herein.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example the invention provides a reagent that comprises a polymerase with RNA dependent DNA polymerase activity and a polymerase with DNA dependent DNA polymerase activity, 0.3% Tween and 8% glycerol. The invention also provides a method of preparing a sample that may comprise a virus for amplification of the viral nucleic acid, where the method comprises heating the sample in a sealed vessel in the presence of a polymerase that has both RNA dependent DNA polymerase activity and DNA dependent DNA polymerase activity to 95 C for 1 second. The invention also provides a method of diagnosing a subject as infected with ebola wherein the method comprises preparing a sample of 2 μl blood taken from the subject for amplification wherein the preparing comprises heating the sample to 95 C for 1 second and directly performing RT-PCR or RT-qPCR on the sample, followed by detection of the amplicon (if present).

### FIGURE LEGENDS

FIG. 1A) shows heating for 1 second at 85, 87, 89, 91, 93, 95C. B) shows heating for 45 seconds at 71/73/74/76/78/80 C. C) heating for 20 seconds at 78/79/80/81/82 C. 81/82 is optimal for this virus—but all temperatures do render the viral nucleic acid amplifiable and to a similar extent. The assay used was the GP14 assay (Trombley A R, Wachter L, Garrison J, et al. Comprehensive panel of real-time TaqMan polymerase chain reaction assays for detection and absolute quantification of filoviruses, arenaviruses, and New World hantaviruses. Am J Trop Med Hyg. 2010; 82(5):954-960. doi:10.4269/ajtmh.2010.09-0636). The target was 100 virions of accuplex ZEBOV control (Seracare LLC, cat no. 0505-001). 50 ul rxn, 800 nm F and R primer, 200 nm Cy5 labelled probe and the reagent as described—50 mM Bicine pH 8.2, 3.4 mM MgCl2, 0.4 mM dNTP, 115 mM potassium acetate, 0.1 μg/μl BSA, 0.2% Tween. 8.5% glycerol, 5 units/rxn of enzyme>95% the same as SeqID 2. The thermal cycling protocol was 85/87/89/91/93/9595 C for 1 second (viral lysis), 90 seconds at 63 C (RT step), 96 C for 3 seconds (hybrid denaturation), 45 seconds 57 C (PCR), 3 seconds at 95 C denaturation—repeated 40 times.

FIG. 2—The methods of the present invention are suitable for use with a variety of sample types. A) shows amplification of nucleic acid from 100 virions in serum (0% blood), and at 9% blood and 15% blood. Surprisingly, the preferred amount of blood is 12-16% and the preferred amount of serum is 12-16%.
B) Direct detection of PPRV virus from cells isolated from lesions of infected animal.
C) Serial dilution of pig nasal swab from an animal infected with PPRV
D) Direct detection of an Ebola pseudovirus from cerebrospinal fluid
E) Whole blood spiked with either a) staphylococcus epidermis b) Escherichia coli at 1000 bacteria/rxn. This demonstrates that bacterial infections in blood could be detected without the need for nucleic acid extraction since the method renders the bacterial nucleic acid amplifiable. F) Direct detection of CCHF from viral culture G) Direct detection of lassa from frozen infected patient samples

For all above reactions the conditions were as follows: 50 ul rxn, 400-800 nm F and R primer, 120-200 nm Cy5 labelled probes and the reagent as described—50 mM Bicine pH 8.2, 3.4 mM MgCl2, 0.4 mM dNTP, 115 mM potassium acetate, 0.1 μg/μl BSA, 0.2% Tween. 8.5% glycerol, 5 units/rxn of enzyme>95% the same as SeqID 2. The thermal cycling protocol was 95° C. for 1 second (viral lysis), 90 seconds at 63 C (RT step), 96° C. for 3 seconds (hybrid denaturation), 45 seconds 57° C. (PCR), 3 seconds at 95° C. denaturation—repeated 40 times. All assays were completed in a closed tube format whereby the sample was added directly into the mix. Experiments E and A were performed in the vessel and method of PCT/GB2019/051156, whereby upon sealing the reaction vessel it was subjected to 500 g for 30 seconds in a centrifuge in order to pull the red blood cells contained therein to the base of the vessel and make plasma in which virions can be directly detected. The appropriate primers and probes were used, for Ebola these were was the GP14 assay (Trombley A R, Wachter L, Garrison J, et al. Comprehensive panel of real-time TaqMan polymerase chain reaction assays for detection and absolute quantification of filoviruses, arenaviruses, and New World hantaviruses. Am J Trop Med Hyg. 2010:82(5): 954-960. doi: 10.4269/ajtmh.2010.09-0636). For CCHF these were from (Barry Atkinson, John Chamberlain, Christopher H. Logue, Nicola Cook, Christine Bruce, Stuart D. Dowall, and Roger Hewson. Vector-Borne and Zoonotic Diseases. September 2012), for the PPRV assay from the following publication (A real time RT-PCR assay for the specific detection of Peste des petits ruminants virus. Journal of virological methods, ISSN: 1879-0984, Vol: 171, Issue: 2, Page: 401-4:2011), for Lassa from assay (Trombley A R, Wachter L, Garrison J, et al. Comprehensive panel of real-time TaqMan polymerase chain reaction assays for detection and absolute quantification of filoviruses, arenaviruses, and New World hantaviruses. Am J Trop Med Hyg. 2010; 82(5):954-960, and lastly for E. coli and s. epidermis the following primers and probes were used:

E.ColiA2_F2
(SEQ ID NO: 5)
GTAACGCGCTTTCCCACC
E.ColiA2_R2
(SEQ ID NO: 6)
TGTGGGCATTCAGTCTGGATC
E.ColiA2_P2
(SEQ ID NO: 7)
CTGATCAATTCCACAGTTTTCGCG
Sep_aap_P04-F
(SEQ ID NO: 8)
CTGTTAATGGGTTCTTAGTTGTTGG
Sep_aap_P04b-R
(SEQ ID NO: 9)
CAAAATATGGTCCAGTTGATGGAGA
Sep_aap_P04-P
(SEQ ID NO: 10)
GTTGTAATTGTTTTTGTTCCTGGTTCACCT

FIG. 3—Tween (polysorbate) allows the amplification components to access the nucleic acid resulting in amplification, whereas Triton X does not.
A) shows a reaction comprising no detergent and minimum solvent which comprised 6% glycerol, 0.1% tween and 6% glycerol, and 0.2% tween and 6% glycerol.
B) shows reactions with Tween at 0.6%, 0.9% and 1.0%.
C) shows a reaction comprising Tween 20 at 0.2% and glycerol at 6% and a reaction at 0.2% Tween 20 with glycerol at 0%
D) shows a reaction comprising Triton X.

FIG. 4A) shows data for the TTH enzyme—9% blood with 1000 virions per reaction. B) shows data for the Hawkz305 enzyme—6% blood at 100 copies per reaction. C) shows data for the enzyme with SEQ ID NO:2.

50 μl reactions, 800 nm F and R primer, 200 nm Cy5 labelled probe and the reagent as described—50 mM Bicine pH 8.2, 3.4 mM MgCl2, 0.4 mM dNTP, 115 mM potassium acetate, 0.1 μg/μl BSA, 0.2% Tween. 8.5% glycerol, 5 units/rxn of enzyme>95% the same as SeqID 2. The thermal cycling protocol was 95 C for 1 second (bacterial lysis), 90 seconds at 63 C (RT step), 96 C for 3 seconds (hybrid denaturation), 45 seconds 57 C (PCR), 3 seconds at 95 C denaturation—repeated 40 times. The reactions were spiked with human blood at 15% final reaction volume.

FIG. 5

Data showing that the reagent of the invention protects against RNA degradation. Reaction performed with a 1 minute temperature hold at 95 C with magnesium at concentrations of 3, 3.2, 3.4, 3.6, 3.8, 4.0 mM. This was performed in a bicine buffer.

FIG. 6—The effect of glycerol on the reaction.
A) shows reactions performed with 6, 7, 8, 9 and 10% glycerol v/v. 8-9% was found to be the best compromise.
B) shows reactions with 0% and 6% glycerol at 0.2% tween20

FIG. 7—The effects of tween on blood sinking.
A) the effect of 0.1%, 0.4% and 1.0% tween on the sinking of blood in Eppendorf tubes.
B) the effect of tween on the sinking of blood in PCR reaction tubes at various concentrations. Before and after PCR images are shown, with an obvious clear layer forming following PCR at higher concentrations of tween which is absent, or minimal, at lower concentrations of tween.
C) amplification data from the reactions set up in B.

FIG. 8—The effects of multiple short holds on reverse transcriptase
A) a single 4 minute reverse transcription step either i) run as a single hold (0 denaturation) or ii) a number of shorter holds that include a number cDNA of denaturation steps, but where the total hold time is equal to that of (i).
B) a set of 15 identical reactions each containing 16% whole human blood and each reaction spiked with 10 viruses-high sensitivity from having multiple chances to initiate PCR at the beginning while the copy number is very low—this is key to meeting the commercially vital 3,000 virions/ml required by WHO for low cost diagnostics.
C) A typical thermal profile for the method—the correct temperatures require determining for each step

FIG. 9A) PCR from blood using a Tet label; and B) PCR from blood using a CY5 label. The reactions were identical save for the probe being labelled by either TET dye or Cy5 and the quencher being BHQ1 or BHQ2 respectively. The target was accuplex ebola reference material. 50 ul rxn, 800 nm F and R primer, 200 nm Cy5 labelled probe and the reagent as described—50 mM Bicine pH 8.2, 3.4 mM MgCl2, 0.4 mM dNTP, 115 mM potassium acetate, 0.1 μg/μl BSA, 0.2% Tween. 8.5% glycerol, 5 units/rxn of enzyme>95% the same as SeqID 2. The thermal cycling protocol was 95 C for 1 second (viral lysis), 90 seconds at 63 C (RT step), 96° C. for 3 seconds (hybrid denaturation), 45 seconds 57 C (PCR), 3 seconds at 96° C. denaturation—repeated 40 times.

FIG. 10—sequence alignment

FIG. 11—Further examples of direct detections from crude samples:
a) Direct detection of Rift Valley Fever virus from an infected mouse model using frozen blood added at 6% of final reaction volume
b) Direct detection of Crimean Congo Haemorrhagic fever virus from viral culture medium.
c) Direct detection of Pest des Petits Ruminants virus from infected pig blood or nasal swabs.
d) Detection of Foot and Mouth virus from viral culture in PBMC. 50 ul rxn, 800 nm F and R primer, 200 nm Cy5 labelled probe and the reagent as described—50 mM Bicine pH 8.2, 3.4 mM MgCl2, 0.4 mM dNTP, 115 mM potassium acetate, 0.1 μg/μl BSA, 0.2% Tween. 8.5% glycerol, 5 units/rxn of enzyme>95% the same as SeqID 2. The thermal cycling protocol was 95° C. for 1 second (viral lysis), 90 seconds at 63° C. (RT step), 96° C. for 3 seconds (hybrid denaturation), 45 seconds 57° C. (PCR), 3 seconds at 95° C. denaturation—repeated 40 times.

The primer sequences are as follows:

CCHF S122F
(SEQ ID NO: 11)
CCTTTTTGAACTCTTCAAACC
CCHF S1R
(SEQ ID NO: 12)
TCTCAAAGAAACACGTGCC
CCHF Probe
(SEQ ID NO: 13)
ACTCAAGGKAACACTGTGGGCGTAAG
RVF Weid F
(SEQ ID NO: 14)
TGCCACGAGTYAGAGCCA
RVF Weid R
(SEQ ID NO: 15)
GTGGGTCCGAGAGTYTGC
RVF Weid
(SEQ ID NO: 16)
PTCCTTCTCCCAGTCAGCCCCAC
PPRVF
(SEQ ID NO: 17)
AGAGTTCAATATGTTRTTAGCCTCCAT
PPRVR
(SEQ ID NO: 18)
TTCCCCARTCACTCTYCTTTGT
PPRVP
(SEQ ID NO: 19)
CACCGGAYACKGCAGCTGACTCAGAA

### EXAMPLES

### Example 1—Temperature Required to Render the Target Nucleic Acid Accessible to the Amplification Components

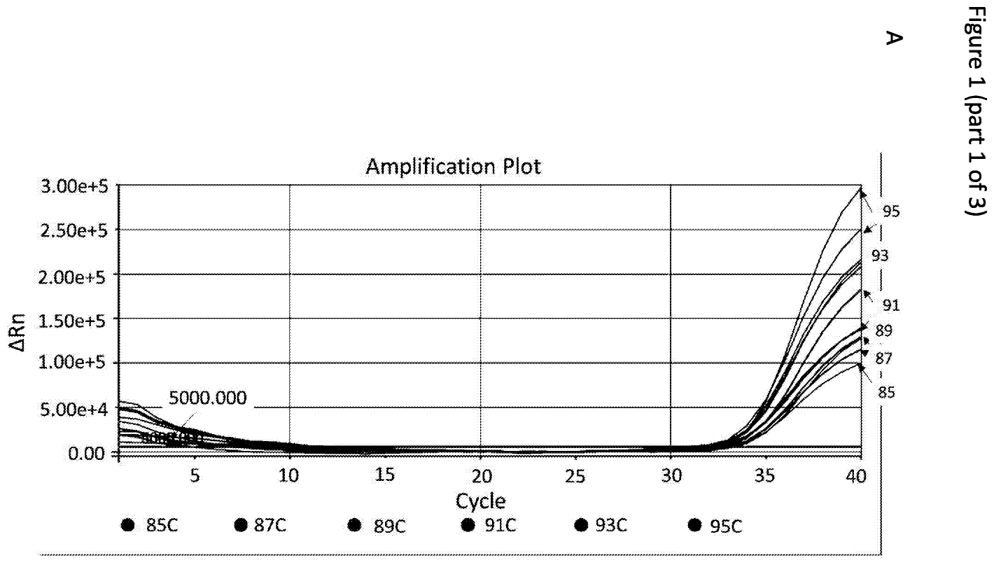

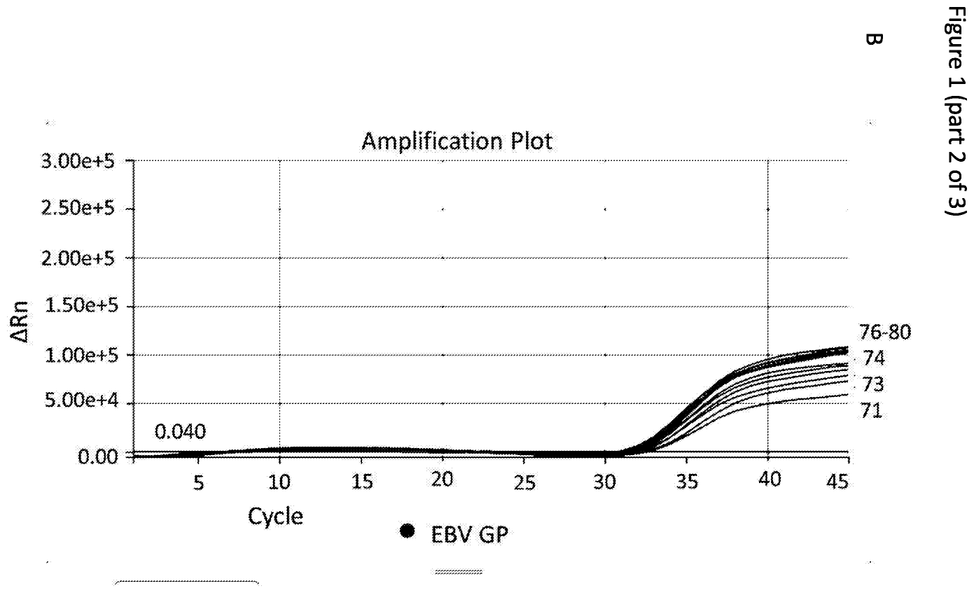

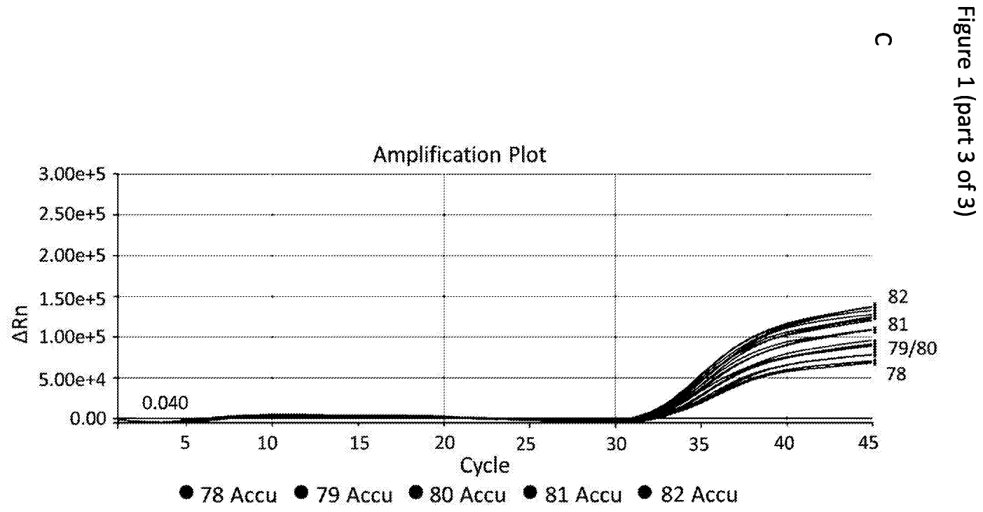

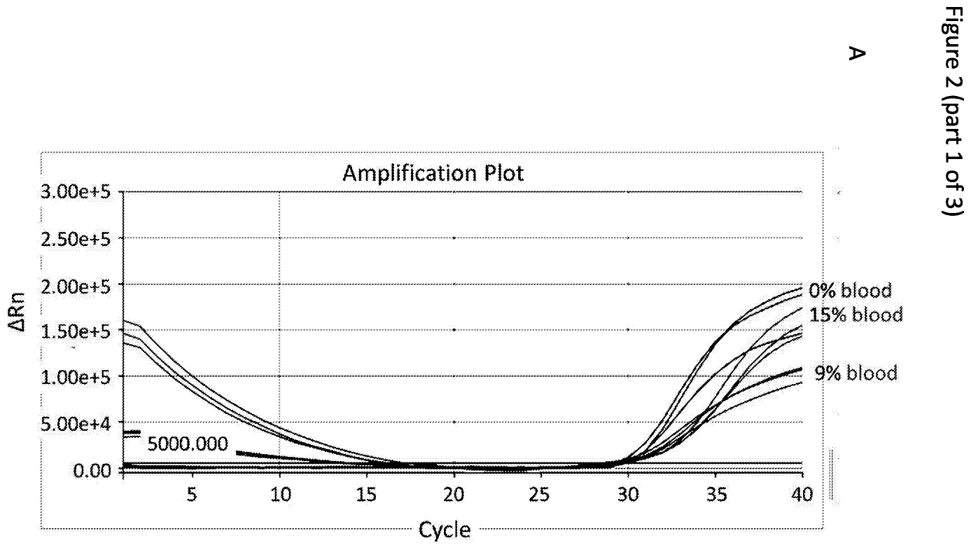

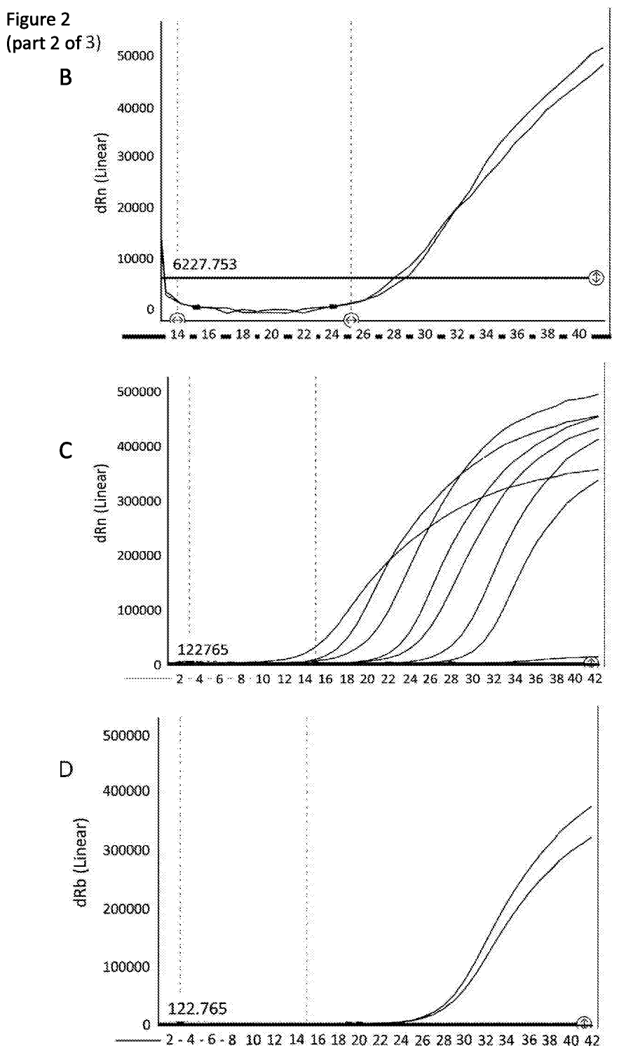

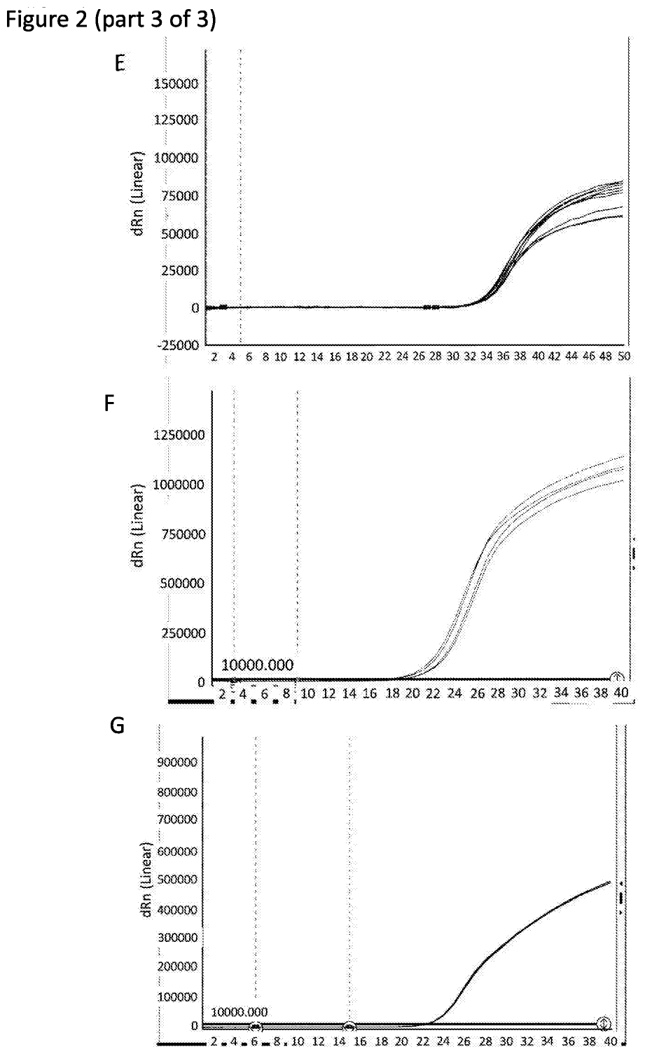

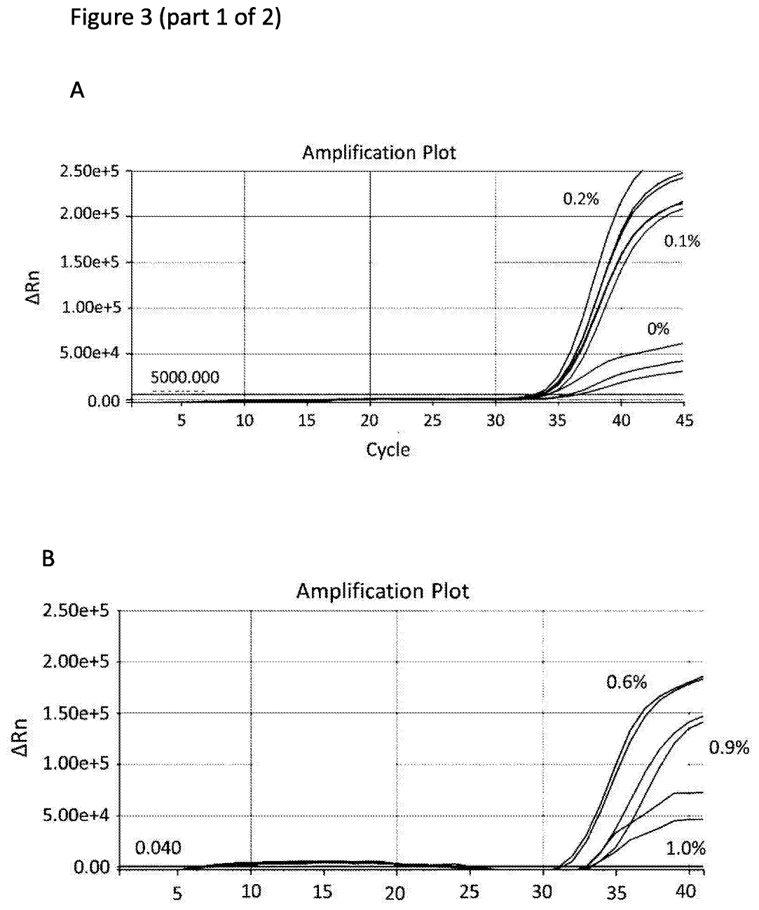

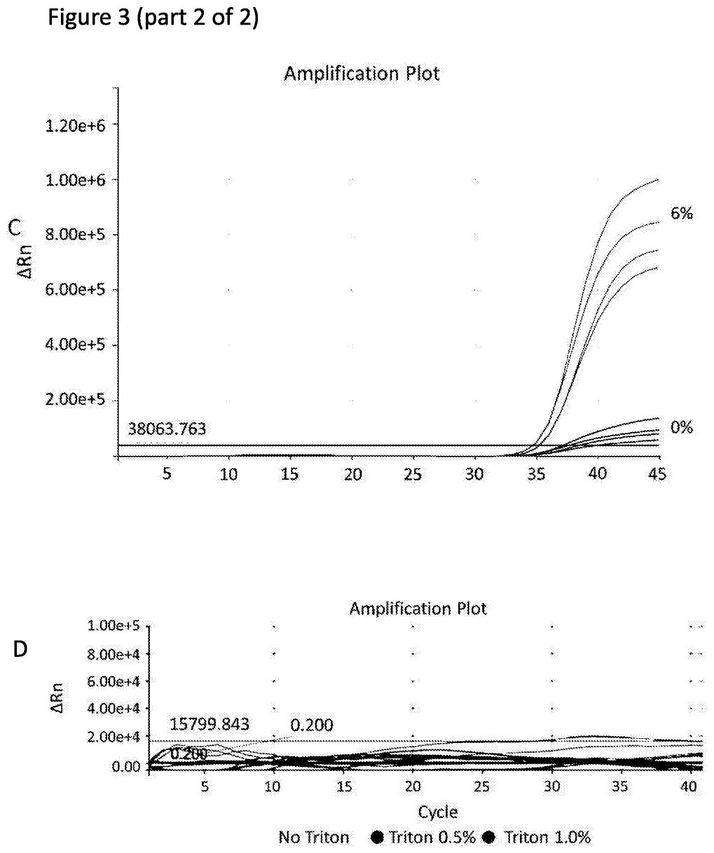

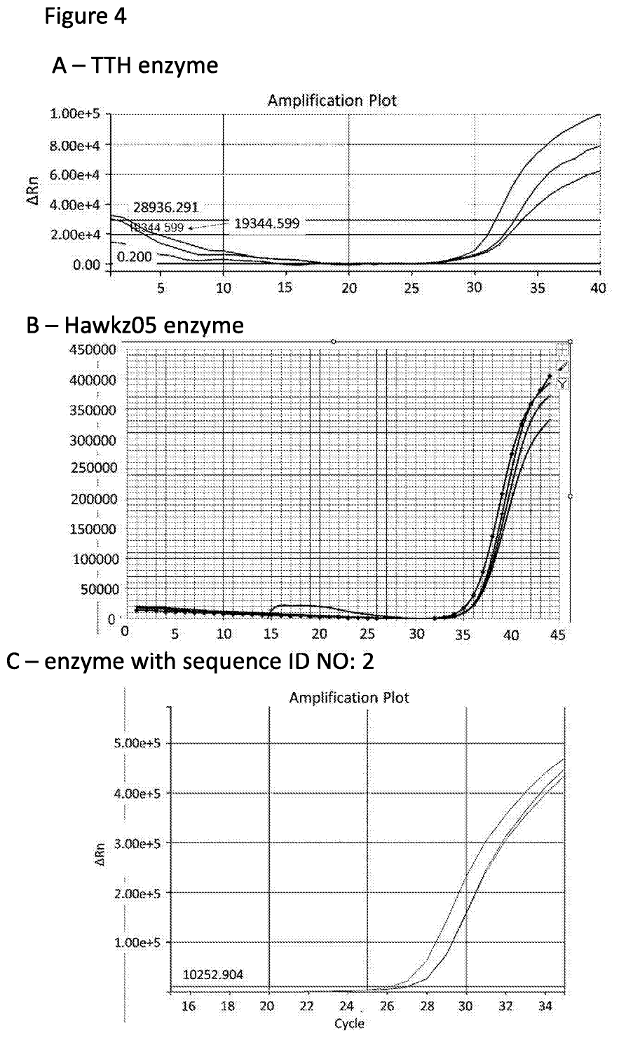

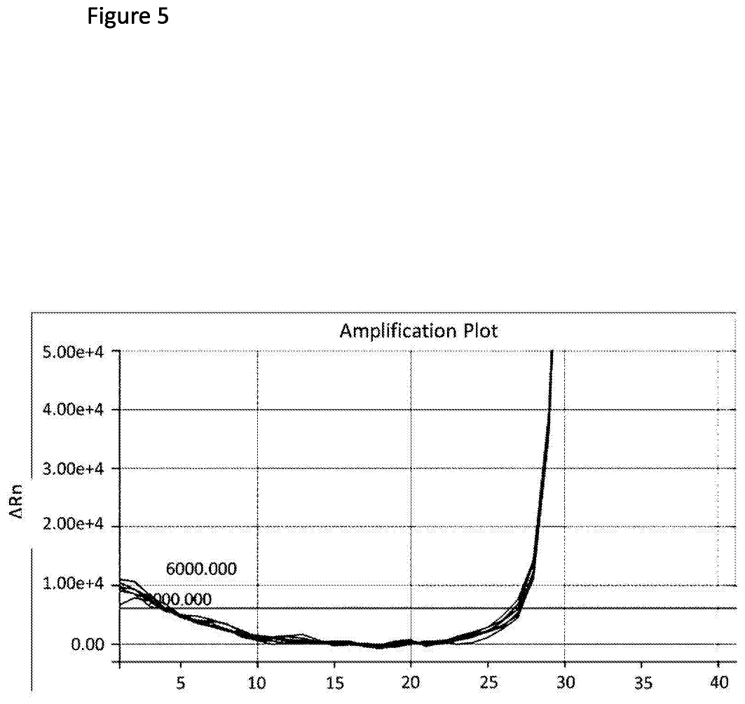

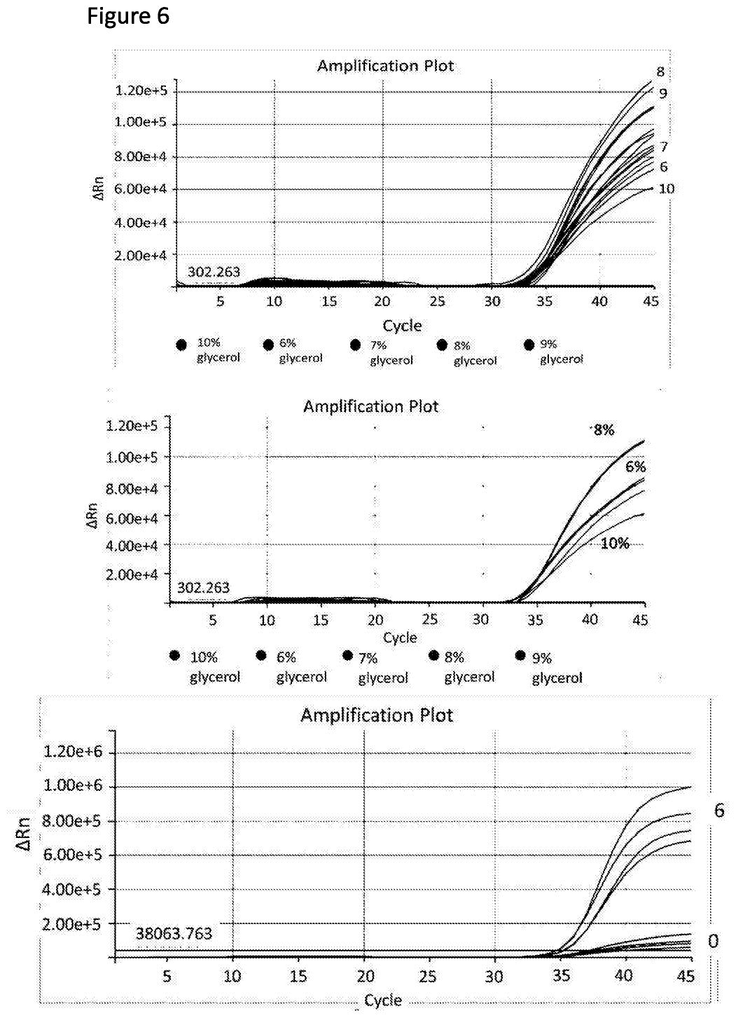

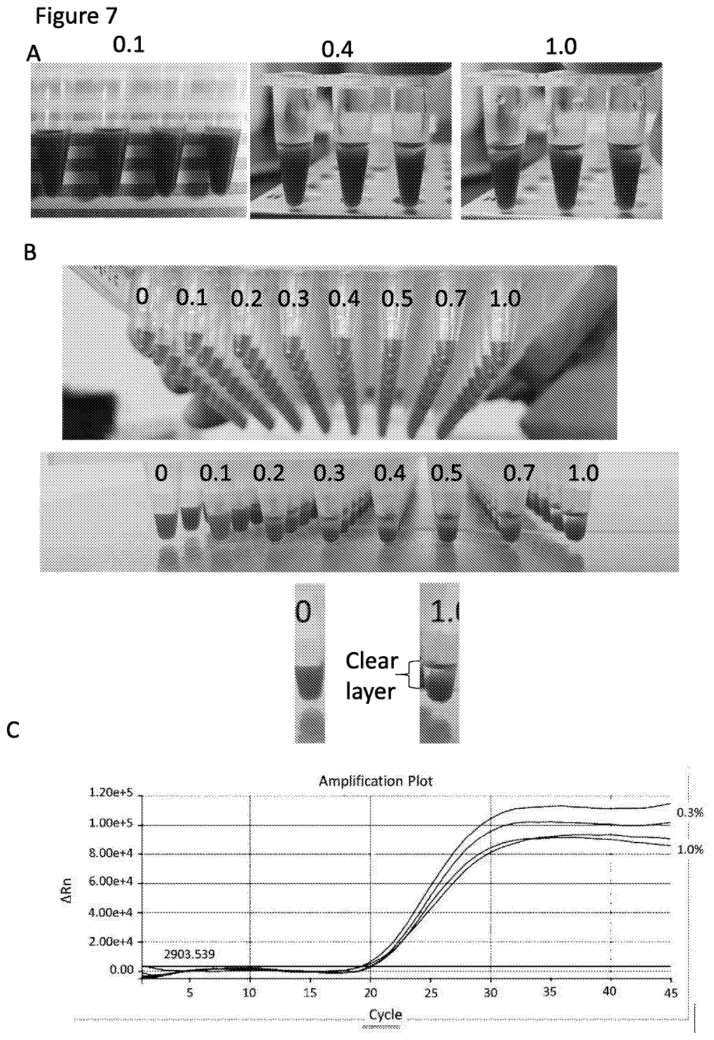

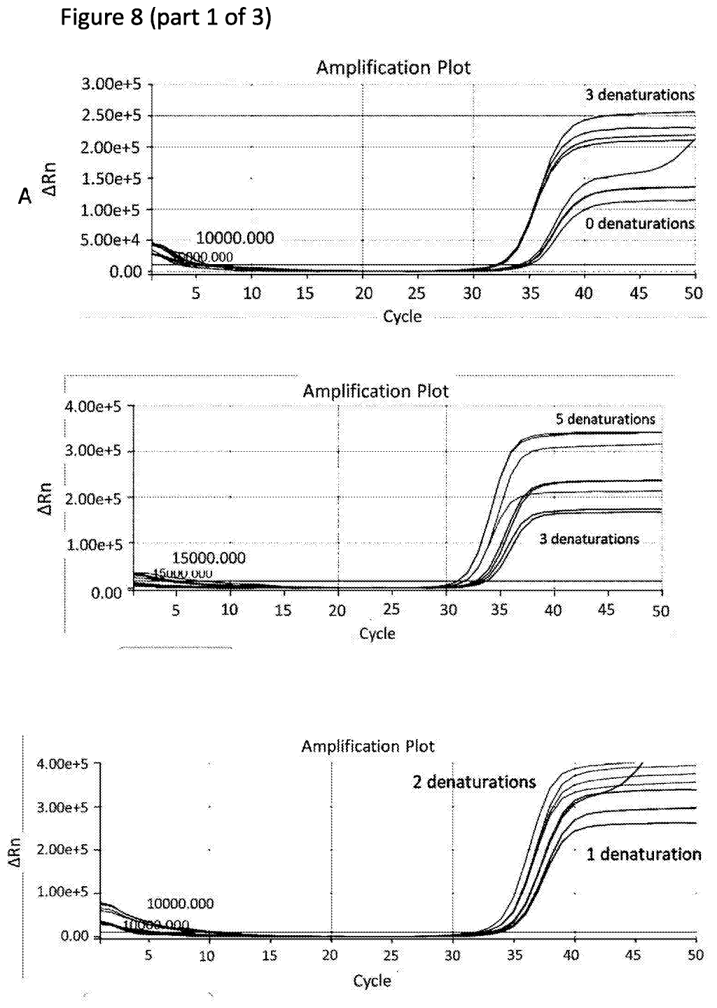

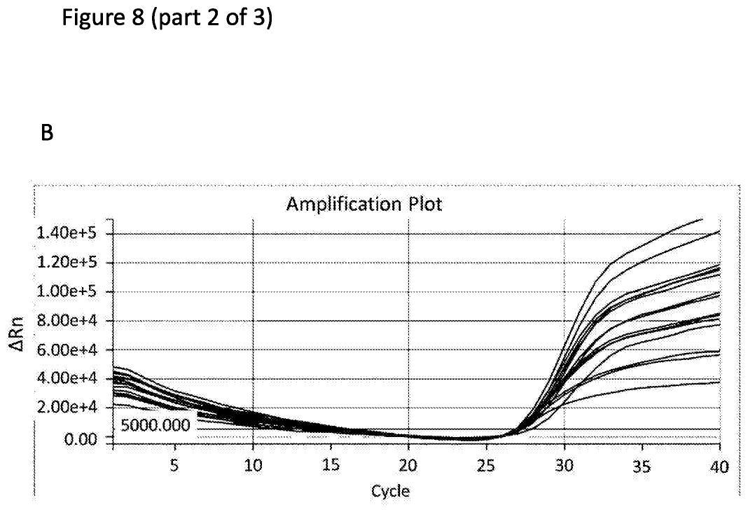

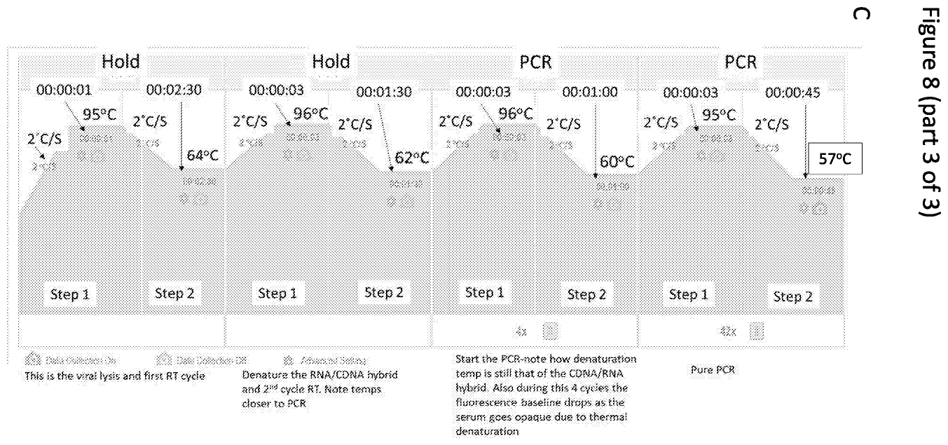

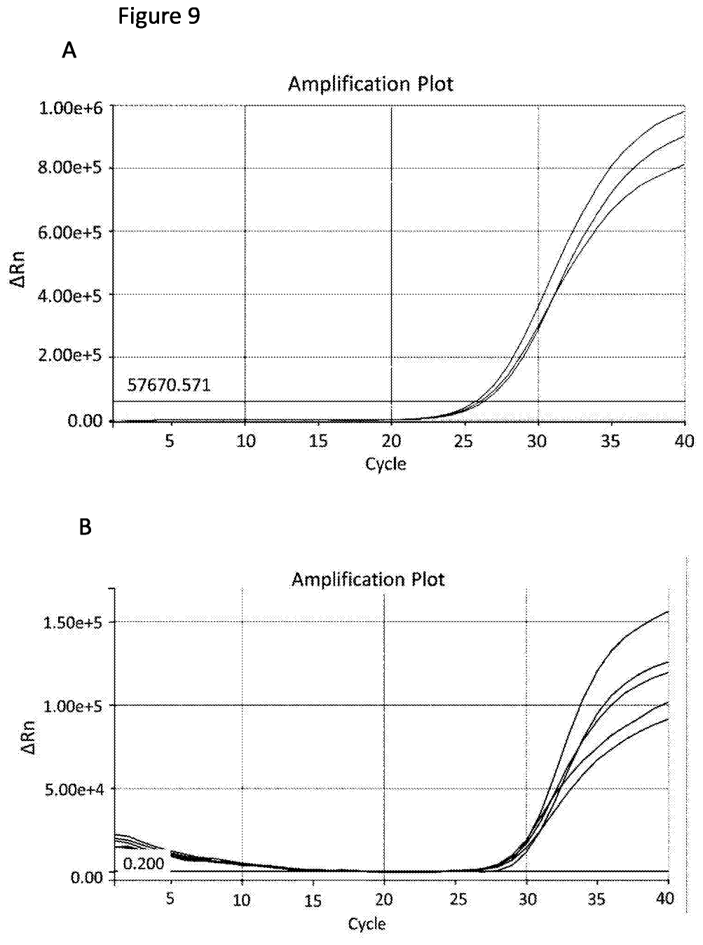

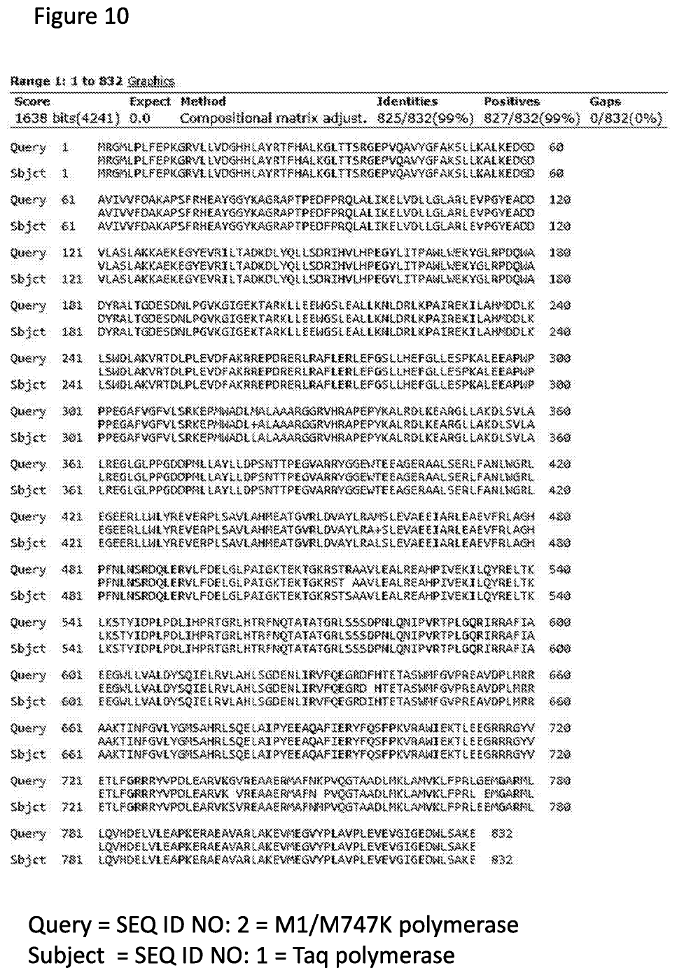

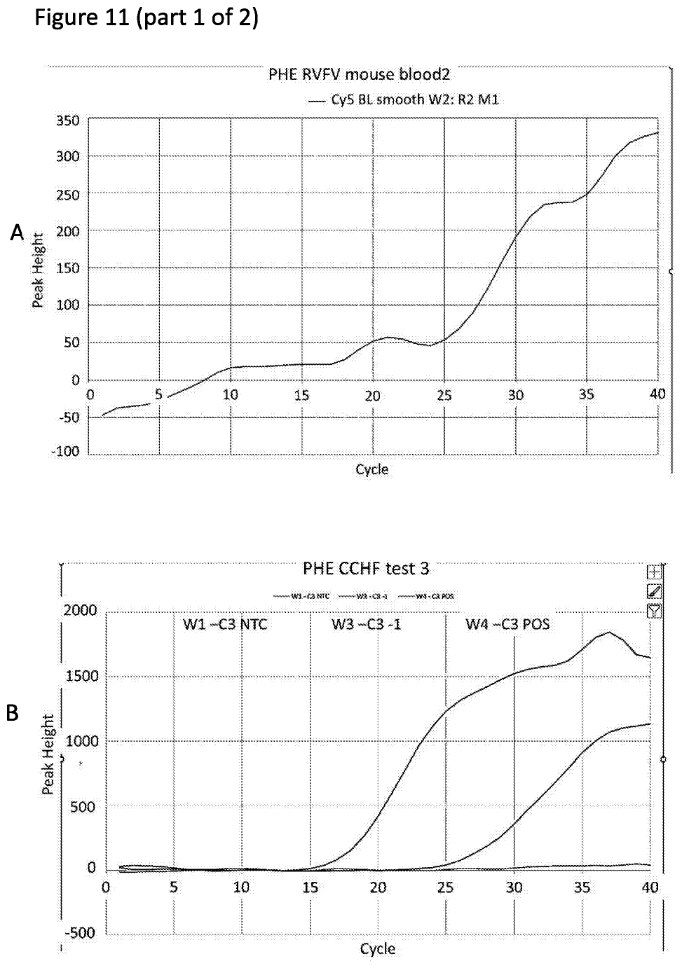

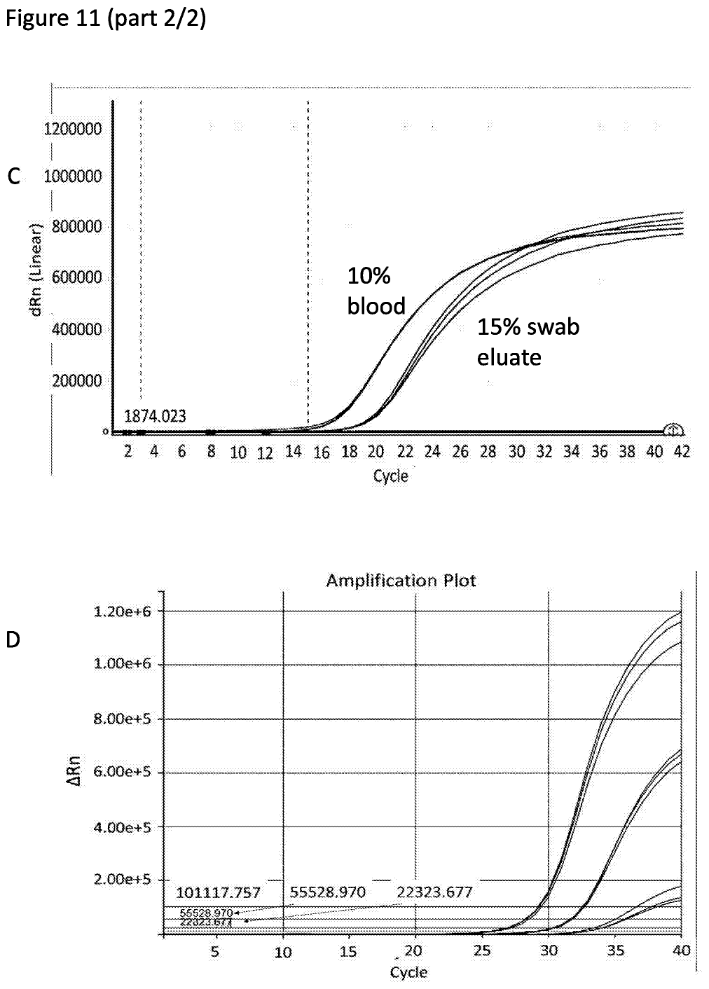

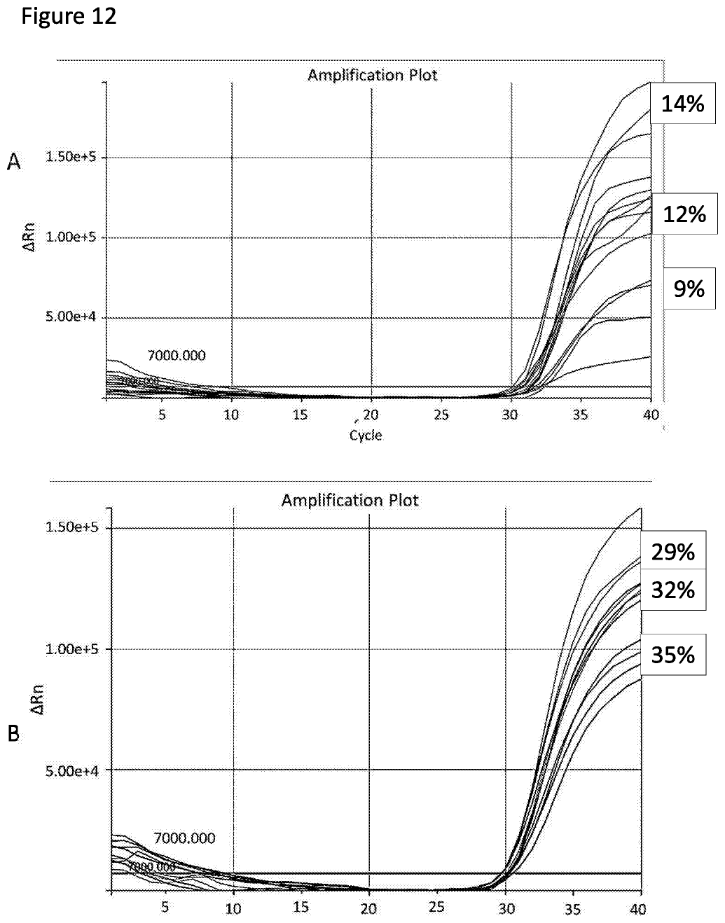

FIGS. 1a, b and c show that heating the sample to a higher temperature for a shorter period of time is preferable to heating to a lower temperature for a longer period of time.

Temperatures over 76 C render viral nucleic acid accessible to the amplification components. However, RNA degrades with time so a shorter hold is preferable, with a short cooler hold being the most preferred. The preferred embodiment is to heat the sample to 93-95° C. for 1 second.

### Example 2—Multiple Sample Types and Viruses/Microbes

The present invention is suitable for use with multiple sample types, including blood and plasma (FIG. 2A), cells from a lesion of an infected animal (FIG. 2B), a pig nasal swab, cerebrospinal fluid, viral culture, frozen infected patient serum samples, and can be used to detect at least Lassa, CCHF, Staphylococcus epidermis, E. coli, ebola pseudovirus, and PPRV. There is no reason to suspect that the present invention cannot be used to detect any virus or microbe that comprises nucleic acid that can be amplified.

### Example 3—the Effects of Tween and Glycerol

A detergent is required to render the viral or microbial nucleic acid amplifiable. Accordingly, heating to above 70 C alone is not sufficient to result in amplification of the nucleic acid. Tween is able to render the viral or microbial nucleic acid amplifiable, but Triton X is not.

Polysorbate 80 is more lipophilic and polysorbate 20 is more hydrophilic so all testing done with tween 20 (polysorbate 20) but there is no reason to suppose that other forms of tween would not also work. It is expected that Triton did not work since the enzyme buffer comprised the detergent Brij and the Brij may not be compatible with Triton. Accordingly detergents other than Tween are considered to be suitable for use with the methods of the invention and the reagent of the invention, though in some instances compatibility with components of the enzyme mixture may need to be assessed. It is assumed that other non-ionic detergents would be compatible with the process and are known in the art.

No detergent means that the target nucleic acid is not accessible to the amplification components, for example since the detergent may be required to alter capsid conformation or remove proteins from the nucleic acid, and leads to lysis and unreproducible data. Addition of anything over 0.1% tween makes the reaction reproducible. As the concentration of Tween gets too high it depresses the tm of the primers/probes. This then damages the PCR itself by reducing PCR efficiency and probing. Practically, in this particular reaction, you cannot use more than 0.7% as it begins this depression

Tween also aids in sinking the sample where the sample is blood. FIG. 7 shows the effect of different concentrations of tween on blood sinking and on amplification. A clear layer forms at the top of tube from 0.1% tween onwards, with the size of the clear layer being tween concentration dependent (the 0% tween tube shows no clear layer at the top at all). However, at higher concentrations, tween lowers the Tm of the primers and lowers PCR efficiency. 0.2-0.3% tween is considered optimal since it provides the clear layer without reducing PCR efficiency.

The solvent and the detergent are both required to be present for efficient and reproducible lysis and amplification to occur

### Example 4 the Method of Invention is Suitable for Use with a Range of Polymerases

FIG. 4A shows data obtained for the TTH enzyme, which has both RNA dependent and DNA dependent polymerase activities. FIG. 4B shows data for the Hawkz05 enzyme which also has both activities.

Although the TTH and Hawkz05 enzymes can be used, they are not as sensitive as the enzyme with 90% or 95% identity to SEQ ID NO: 2 since TTH performs less well as a reverse transcriptase and Hawkz05 is more inhibited by the presence of blood.

### Example 5 RNA Protection by the Reagent

It is well known that the combination of Tris and Mg results in nicked RNA (AbouHaidar and Ivanov, 1999 Z Naturforsch 54: 542-548).

The methods and reagents of the present invention minimise RNA degradation.

The best evidence of low RNA degradation is consistent performance across a range of magnesium concentrations and hold times. FIG. 5 shows data from a 1 minute heating step at 94 C with magnesium at 3, 3.2, 3.4, 3.6, 3.8, 4.0 mM. This is a relatively long time at a high temp, with high Mg²⁺ but the Ct is identical. This indicates that there is no difference in RNA degradation across a 25% increase in MgCl₂. A possible mechanism, and hence reason for the selection of Bicine/tricine, is that they are known to be able to buffer the amount of free metal ions available free in solution and that this plus the lowered pH will minimise hydroxyl attack of the RNA (Free-metal ion depletion by “Good's” buffers R Nakon, CR Krishnamoorthy, Science 19 Aug. 1983: Vol. 221, Issue 4612, pp. 749-750 DOI: 10.1126/science.6879173).
this means that the RNA stays in the reaction and so is available for multiple cycles of reverse transcription, if necessary.

The buffers of the invention are, in some embodiments, also pH neutral at the temperature above 70 C to which the sample is subjected.

[symbol]

Temperature Dependence of pH for
Commonly Used Buffers.

| Buffer | | |
|---|---|---|
| System | pK [symbol] /20° C. | ΔpK [symbol] /10° C. |
| MES | 6.15 | −0.110 |
| ADA | 6.60 | −0.110 |
| PPES | 6.80 | −0.085 |
| ACES | 6.90 | −0.200 |
| BES | 7.15 | −0.160 |
| MOPS | 7.20 | −0.013 |
| TES | 7.50 | −0.200 |
| HEPES | 7.55 | −0.140 |
| Tricine | 8.15 | −0.210 |
| Tris | 8.30 | −0.310 |
| Bicine | 8.35 | −0.180 |
| Glycylglycine | 8.40 | −0.280 |

Reference
Good, N.E. (1986) Biochemistry 5, 467. [symbol]
indicates data missing or illegible when filed

Bicine drops 0.18 pH units/C according to the published data.

So at 95° C. a bicine buffer is pH 7.0 and so no RNA attack can take place during the denaturation step as this needs an excess of hydroxide ions.

Bicine buffers are considered to be particularly preferred since it was the only buffer in which all 3 enzymes (TTH, Hawk305 and the enzyme with SEQ ID NO: 2) were shown to workTth will do PCR in Tris buffers but only RT in bicine. Hawk305 will work in bicine or tricine so likely tricine can be substituted for bicine. Bicine buffers were also shown to help RNA survive by being immune to high temperatures and MgCl₂ concentrations.

### Example 6—Repetitive Reverse Transcriptase

The inventors of the present invention have found that it is possible, and beneficial, to perform reverse transcriptase as a series of shorter holds that include a cDNA denaturation step, rather than a single longer hold of equivalent time.

FIG. 8 shows data for a single 4 minute reverse transcription step either run as a single long hold, or a series of shorter holds broken up by a cDNA denaturation step. Each reaction is run for the same overall length of time.

Note the higher final fluorescence for having performed more RT cycles within the same fixed time period—this indicates more RNA was turned into CDNA.

The reactions contained 250 virions and 9% whole human blood.

5 denaturations produced the earliest Ct—so 6×41 second RT steps taking the same time as 1 4 minute RT step improved the data because the RNA survives and new CDNA is made each cycle.

RNA to DNA bonds are energetically stronger than DNA/DNA bonds, as a practical example the primer sequence GATACACTGGGATGACTCTTTGCCGAAC (SEQ ID NO:20) has a Tm to DNA of 71° C. but to the target RNA of 76° C.

This means that running the reverse transcription at 76° C. would give a successful RT reaction but produce very little PCR product if multiple cycles were performed due to only 1.74% of the primer being able to bind at an annealing temperature some 5 C above it's Tm to DNA.

Therefore it is possible by performing multiple high temperature RT steps to make multiple cDNA molecules from the same RNA strand and hence increase sensitivity by having this pre-amplification step. The described reagent ensures that the RNA molecules survive long enough at the elevated temperatures such that multiple cycles can be achieved.

An alternate strategy is to design Primers where the Tm to RNA and DNA are much closer together by the use of locked nucleic acids (LNA). LNAs are an RNA species whose confirmation is locked in the 3′-endo confirmation which has the effect of stabilising intra strand binding. As a result, the Tm of the forward and reverse to DNA can be brought closer together while the LNA moiety lifts the Tm to the RNA target. It is 20 then possible to design assays whereby the RT and PCR can be efficiently be performed at the same temperature-PCR is an amplification chemistry based on a doubling of the initial target copy number as opposed to the simple single copy transcription or reverse transcription. Sensitivity will therefore be maximised if both can be efficiently performed at the same temperature-since an efficient PCR will be initiated at the same time the RT function is still providing initial opportunities for the initial low copy number of RNA targets to be turned into a cDNA which can then be amplified and detected.

As the reagent minimises RNA degradation it means that the target nucleic acid such as viral RNA remains in solution and each cycle gives a further chance for RNA to be turned into a cDNA.

FIG. 8B shows a set of 15 identical reactions each containing 16% whole human blood and each reaction spiked with 10 viruses. The high sensitivity comes from having multiple chances to initiate PCR at the beginning while the copy number is very low—this is key to meeting the commercially vital 3,000 virions/ml required by WHO for low cost diagnostics. In this instance the primers are designed to allow both RT and PCR to occur simultaneously. Such an approach was found to be the most sensitive.

The disadvantage to using an enzyme that can perform RT and PCR is that it is very hard to separate the two functions. To determine the optimum temperatures for the repetitive RT it is necessary to perform a comparison of the detection sensitivity across a range of temperatures (see FIG. 8C). The higher the temps and the longer the holds the more inhibitors are potentially released from the sample, such as blood, so you want to minimise temps and holds as much as you can-which also speeds up the time to detection.

### Example 7 the Methods Work with any Fluorophore that Emits and Excites at any Wavelength

Viral nucleic acid from a sinbis virus expressing the Ebola genome (Accuplex Seracare) was amplified from a blood sample, and labelled with a Tet label (FIG. 9a) or a CY5 label (FIG. 9b). The Tet label excites at around 521 nm and emits at around 536 nm; whilst the CY5 label excites at around 625 nm or 650 nm and emits at around 670 nm. Accordingly, since the methods and reagents are suitable for the detection of amplicons labelled with both red and green fluorophores, which are at opposite ends of the spectrum, the invention is considered to be suitable for use with any fluorophore.

### Example 8—Preferred Embodiment

The optimal reaction conditions that are applicable to a wide range of sample types/viruses/microbes were found to be 50 mM Bicine, 3.4 mM MgCl₂, 115 mM Potassium Acetate, 8% glycerol, 0.4% tween at pH 8.2 at 25° C. A range of MgCl₂ concs from 3-4 mM MgCl₂ generate amplicon but 3.4 mM MgCl₂ proved to be optimal. Preferably the enzyme M1/M747K is used (SEQ ID NO: 2).

The process for detecting viral pathogens direct from crude samples in a closed tube assay may be as follows.

Add the crude sample to the reaction vessel containing 50 mM Bicine, 3.5 mM MgCl2, 115 mM Potassium Acetate, 8% glycerol, 0.4% tween at pH 8.2 at 25° C. and the enzyme, preferably M1/M747K or an enzyme with at least 90% or at least 95% identity to SEQ ID NO:2, primers specific to the target(s) of interest and sequence specific fluorescent probes.

Raise the temperature of the reaction vessel to 95° C. for 1 second. (renders the viral/microbial nucleic acid to be accessible to the amplification components) Drop the temperature to that optimal for the reverse transcription step as determined by the primer sequence specific to the target of interest.
Denature the target at 96 C for 3 seconds
Optionally perform multiple rounds of repetitive reverse transcription
Perform QPCR amplification from the resulting cDNA and detect the resultant amplicon.
   This preferred thermal cycling protocol is shown in FIG. 8C

| Buffer | 1× | |
|---|---|---|
| MgCl2 | 0.4 | mM |
| BSA | 0.1 | ug/uk |
| dNTPs | 0.4 | mM |
| Tween | 0.2% | |
| Enzyme | 5 U/50 ul | |
| forward primer | 1 | uM |
| reverse primer | 1 | uM |
| Probe | 0.25 | uM |

Nuclease-free water

| | |
|---|---|
| Bicine-pH 8.2* | 50 mM |
| Glycerol | 9% v/v |
| MgCl2 | 3.4 mM |
| Potassium Acetate | 115 mM |
| Bovine Serum Albumin | 0.1 mg/ml |
| Tween | 0.2% vol/vol |
| Tris-HCl-pH9.2* | 1 mM |
| Ammonium Sulphate | 0.32 mM |

| | | | Potassium | Magnesium | Magnesium | |
|---|---|---|---|---|---|---|
| Buff- | | Bicine | Acetate | acetate | chloride | pH at |
| er | conc | (mM) | (mM) | (mM) | (mM) | 25° C. |
| A | 10x | 500 | 850 | | 25 | 8.0 |
| B | 10x | 500 | 850 | | 25 | 7.8 |
| C | 10x | 500 | 850 | 25 | | 8.0 |
| D | 10x | 500 | 850 | 25 | | 7.8 |
| E | 10x | 500 | 850 | | 28 | 8.2 |
| F | 10x | 500 | 1150 | | 39 | 8.2 |
| G | 10x | 500 | 850 | | 30 | 8.2 |
| H | 10x | 500 | 950 | | 30 | 8.2 |

### Example 9

This specification describes reagents and methods that make possible the direct amplification of pathogen nucleic acids, and is particularly concerned with RNA viruses, direct from whole blood samples suspected of being infected. The method can also be applied to bacterial and fungal pathogens and from a wide range of input samples including serum, plasma, urine, cerebrospinal fluid, faeces and swabs taken from the eyes, nose and mouth.

Additionally, described are methods that enable maximal sensitivity to be established by subjecting the reaction to multiple rounds of reverse transcription where the target pathogen is a virus and a methodology to determine the point at which point individual viral pathogens will lyse and hence be detected.

The specification includes the methods necessary to perform the direct amplification of viral pathogens from crude whole blood samples in a single closed tube process without recourse to performing nucleic acid extraction, making rapid low-cost in-field diagnostics possible.

A number of methods have been shown to be suitable for the inactivation of viral pathogens, including UV, solvent/detergent and heat. Thermal processing, is typically used for the inactivation of viral pathogens in biologically active substrates such as blood and blood products, where gently heating to in the region of 60 C has been demonstrated to render pathogens such as HIV and HepC none infectious. Recent research (Fry et al. A plate-based high-throughput assay for virus stability and vaccine formulation, Journal of Virological Methods, Volume 185, Issue 1, 2012, Pages 166-170) called the PaSTRy technique has suggested that this thermal inactivation is a 2 stage process whereby at a lower temperature (Tr) the viral genome is released from the capsid and a higher temperature (Tm) the capsid itself is degraded. The virus is rendered non-infectious by either of these processes. The authors invented this process for the production of vaccines, for any given virus it is possible to determine when genome release occurs and yet the capsid itself is still intact and hence make attenuated vaccines more reliably. The authors of this specification have discovered that the Tm point coincides with the point upon which amplifiable viral nucleic acid is freed, at the Tr point some proteinaceous component, presumably nucleoproteins remain collocated with the viral genome that make it impossible to be amplified by molecular biological methods such as isothermal amplification or PCR. It is advantageous to have a method of rendering viral pathogens amplifiable direct from crude samples, as normally a time consuming extraction process is required, during which a skilled operative is required and that the operative may be exposed to the deadly Pathogen.

The authors of this specification proposed that if it were possible to add a crude sample into a reaction vessel suspected of containing a viral pathogen and raising the temperature to the Tm point of a virus, that it may be possible to directly amplify the viral genome released therein. The applicants (BG Research) have previously described a method for the closed tube lysis of pathogens to release amplifiable nucleic acids (EP 2585581), the approach here has the benefit of reduced energy requirements, since it is not necessary to freeze and additional is more rapid than multiple cycles of freezing.

The applicants have performed the PaSTRy approach on a number of pathogens including Dengue and have discovered that the Tm of all important pathogens tested to date lies in the range of 74-84° C. Therefore, if a single short hold is added to a nucleic acid amplification process in the range of 74-84 C it was theorised that direct amplification of the contained viral pathogen might be possible. It should be noted that the majority of important deadly pathogens including Ebola, Lassa, SARS and others posses positive sense strand RNA genomes and as such a reverse transcription step is important to successfully amplify the pathogen. The majority of native reverse transciptases in the literature such as MMULV and more modern modified enzymes will be denatured by these higher temperatures necessary to denature the viral capsid at the Tm point. Therefore, the process described in this specification requires the use of thermostable enzymes such as Bioneer Rocketscript that can survive a short excursion to these temperatures.

An additional issue is the thermal stability of the released RNA itself, RNA degrades in the presence (Non-Enzymatic RNA Hydrolysis Promoted by the Combined Catalytic Activity of Buffers and Magnesium Ions) of divalent cations in a basic solution. As a result if the process described here is used with a standard Taq polymerase buffer based on Tris and 4M MgCl2 the released RNA is rapidly degraded and the target pathogen RNA is rendered non-amplifiable. To overcome this the applicants have developed buffers that are substantially pH neutral at the capsid Tm temperature and that buffer divalent cations such that the free concentrations are minimised, based on bicine and tricine—a typical formulation might be 50 mM bicine/tricine, 3.5 mM MgCl2, 115 mM potassium acetate and adjusted to pH 8.2 at 25° C., this buffer will be essentially neutral at the Tm (capsid denaturation point) and as such there will be no excess hydroxyl ions to attack the RNA and yet the pH will be at the physiological requirement for the enzyme at the reverse transcription/amplification step 7.4-7.6.

The applicants have observed that the Tm point can be lowered by the addition of solvent/detergent to the amplification reagents mixture, many solvent detergent combinations are used commercially for viral inactivation, although these were found to largely be incompatible with numerous amplification approaches. A suitable combination was found to be Glycerol at 8% and Tween 20 at 0.1-0.4%. For Tween the percentage added does further drop the TM point, but the applicants have also observed that the hybridisation temperature of any primers and probes, such as for the PCR process, was depressed by higher concentration of Tween that 0.4%

The optimal reaction conditions were found to be 50 mM Bicine, 3.5 mM MgCl2, 115 mM Potassium Acetate, 8% glycerol, 0.4% tween at pH 8.2 at 25° C.

The process for detecting viral pathogens direct from crude samples is as follows.

Add the crude sample to the reaction vessel containing 50 mM Bicine, 3.5 mM MgCl2, 115 mM Potassium Acetate, 8% glycerol, 0.4% tween at pH 8.2 at 25° C. and an enzyme suitable for performing reverse transcription and cDNA amplification/detection
Raise the temperature of the reaction vessel to 76-81 C for 15-60 seconds. (viral lysis step)
Drop the temperature to that optimal for the reverse transcription step
Perform amplification from the resulting cDNA.

76-81° C. has proven to be the correct range for Tm (capsid denaturation) for all viruses tested to date, temperatures in excess of this lead to rapid RNA degradation and hence reduced sensitivity.

### Veterinary Detection Method

In the veterinary field the most universally used sample type is the swab—these being taken from either the eyes, the nose or the mouth dependent upon the disease suspected. A number of virulent animal pathogens, such as Rinderpest and PPRV, have a viraemic component but for some economically important diseases there is a very limited time window that the viral pathogen can be found in the blood. The applicants have described a method for the direct detection of whole blood and this has been shown to be suitable for the detection of the acute viraemia for diseases such as foot and mouth and PPRV (Kavit Shah, Emma Bentley, Adam Tyler, Kevin S Richards, Ed Wright, Linda Easterbrook, Diane Lee, Claire Cleaver, Louise Usher, Jane E Burton, James Pitman, Christine B Bruce, David Edge, Martin Lee, Nelson Nazareth, David A Norwood, Sterghios Athanasios Moschos. Field-deployable, Quantitative, Rapid Identification of Active Ebola Virus Infection in Unprocessed Blood. Chem. Sci., 2017: DOI: 10.1039/C7SC03281A)—however, there are a number of disadvantages to using a direct from blood approach in the remote, resource poor environments where these diseases are endemic. Firstly, the taking of a blood sample from an animal requires the input of a trained veterinarian and secondly that the virus can be found in easier to access samples that can be taken by the lay person. This application covers a method for performing direct detection of viral animal pathogens direct from swab samples taken from the mouth, eyes or nose and without recourse to nucleic acid extraction.

### Example 10

Further demonstration of the utility of the method for detecting viral pathogens for real world crude samples is the ability to detect a range of important viral pathogens. FIG. 11 (A) shows the direct detection of Rift Valley Fever virus from an infected mouse, (B) the detection of Crimean Congo Haemorrhagic fever virus direct from viral culture and the detection of (C) Peste des Petits Ruminants virus direct from either blood or nasal swab samples. This demonstrates the utility of the approach for creating rapid, low-cost diagnostics for emerging diseases impacting animals and humans. The ability to work from swabs means that respiratory diseases can be easily identified and the technology has the ability to detect the WHO top 10 list of viral pathogens and has already been used to generate proof of concept data for ebola, lassa, dengue, rift valley fever, Marburg, Crimean congo fever as well as important veterinary diseases such as foot and mouth (D) and PPRV which cause massive economic impact in the developing world while having commercial utility globally to screen animals at penside and crucially, at the ports.

### Example 11

Demonstrating that in conjunction with the vessels and teachings from PCT/GB2019/051156 it is possible to detect viral pathogens in whole blood at up to 3% final volume per reaction, giving a very high sensitivity and meeting the technical goal of direct from blood detection that meets the WHO R&D blueprint goal of 3,000 virions/ml. FIG. 8B shows 15 identical reactions at 1042 virions/ml, easily meeting the sensitivity requirement and demonstrating the utility of the approach. FIGS. 12A and B demonstrate that the reagent and method work reliably at up to 35% whole blood in the reaction volume, interestingly there seems to be an improvement in sensitivity in the range of 12-16%. This is assumed to be as a result of molecular crowding (Sasaki, Y., Miyoshi, D., & Sugimoto, N. (2006). Effect of molecular crowding on DNA polymerase activity. Biotechnology Journal, 1(4), 440-446), the point at which the maximum benefit of molecular crowding is achieved while minimising inhibition from blood components must correspond with this 12-16% range. Practically this means that a 95 μl reaction containing 15-μl of whole blood is at circa 16% and FIG. 8B has 10 copies/rxn and hence a sensitivity of 1042 virions/ml.

1. A method of preparing a sample obtained from a subject that may contain one or more microbes or viral particles for the direct amplification of the microbial or viral nucleic acid by a polymerase, wherein the method comprises heating the sample in a vessel to a temperature of at least 70° C. in the presence of a reagent that comprises a detergent, a solvent and one or more nucleic acid polymerases.

2. The method of claim 1 wherein (a) the microbial or viral nucleic acid is not extracted, optionally wherein the microbial or viral nucleic acid is not precipitated with an alcohol, optionally is not precipitated with ethanol; (b) the temperature of the sample is not reduced relative to the ambient temperature, optionally wherein the sample is not frozen; (c) the sample is not centrifuged following: i) before heating; and/or ii) following said heating; (d) no part of the sample or reagent is removed from the vessel: i) prior to said heating; and/or ii) following said heating; optionally wherein once the sample is added to the vessel, no material is removed from the vessel; and optionally, wherein the sample is a crude sample, optionally wherein the sample is a sample of: i) blood; ii) urine; iii) serum; iv) plasma; v) faeces, vi) cerebral spinal fluid; vii) a swab, optionally a swab from the eyes, ears, nose or mouth; and/or viii) eluate taken from a wash of a swab.

3. (canceled)

4. (canceled)

5. (canceled)

6. (canceled)

7. The method of claim 1 wherein: (a) the sample comprises particulate or cellular matter; (b) the sample is a mammalian sample, optionally a human, cattle, swine, cow, sheep, pig, dog, camel, horse, llama, goat, rabbit, cat, rat mouse, ferret, guinea pig, mink, or other model organism, or is an avian sample or is a fish sample; (c) the one or more microbes or viral particles may be selected from: i) the group consisting of viruses, bacteria, fungi; and/or ii) class 4 or 3 pathogens; and/or (d) pathogens that cause the diseases selected from the group consisting of: i) Viral haemorrhagic fevers selected from the group consisting of Ebola, Lassa fever, Marburg virus disease, Rift valley fever, Congo fever and yellow fever; and/or ii) Japanese encephalitis, Dengue, Zika, Chikungunya; iii) Veterinary diseases with a viraemic component, including but not limited to PPRV, FMDV, BTV, Newcastle disease, Swine Flu, BVDV iv) Malaria, HIV, viral hepatitis, soil transmitted helminth parasitic infections; and/or (e) the sample comprises a substance, or releases a substance, that is inhibitory to PCR, optionally releases a substance upon heating that is inhibitory to PCR; and optionally, wherein the viral particle is a viral particle that comprises an RNA genome.

8. (canceled)

9. (canceled)

10. (canceled)

11. (canceled)

12. The method of claim 1 wherein: (a) the reagent is compatible with the one or more polymerases; (b) the reagent is compatible with the reagent in which the one or more polymerases is supplied; (c) the reagent minimises RNA degradation, optionally minimises the non-specific catalysis of RNA by magnesium cations; (d) the reagent does not comprise Tris, (e) the reagent comprises bicine, optionally between 20 mM and 70 mM Bicine, optionally between 25 mM and 65 mM Bicine, optionally between 30 mM and 60 mM Bicine, optionally between 35 mM and 55 mM Bicine, optionally between 40 mM and 50 mM Bicine, optionally 50 mM Bicine; (f) the reagent buffers divalent cations.

13. (canceled)

14. (canceled)

15. (canceled)

16. (canceled)

17. The method of claim 12 wherein the reagent comprises tricine and/or the solvent is glycerol and/or the detergent is Tween, optionally Tween 20.

18. (canceled)

19. (canceled)

20. The method of claim 1 wherein: (a) the reagent comprises Tween, optionally Tween20 at a concentration of up to 0.4%, optionally comprises Tween at a concentration of:
a) at least 0.025%, 0.05%, 0.075%, 0.1%, 0.15%, 0.2%, 0.25%, 0.3%, 0.35%, 0.4%; and/or
b) less than 0.4%, 0.35%, 0.3%, 0.25%, 0.2%, 0.15%, 0.1%, 0.075%, 0.05%, 0.025%; and/or
c) between 0.025% and 0.4%, 0.05% and 0.35%, 0.075% and 0.3%, 0.1% and 0.25%, 0.15% and 0.2%; optionally wherein the concentration of tween is between 0.15% and 0.4%;
(b) the reagent comprises glycerol at a concentration of up to 11%, optionally wherein the reagent comprises 11%, 10%, 9%, 8%, 7%, 6%, 5%, 4%, 3%, 2%, 1%, 0.5% or less glycerol, optionally wherein the concentration of glycerol is between around 8%-9%;
(c) the reagent is at a pH of between around 6.5-7.5 at a temperature of between around 70° C.-100° C., optionally between 72° C. and 98° C., 74° C. and 96° C., 76° C. and 94° C., 78° C. and 92° C., 80° C. and 90° C., 82° C. and 88° C., or to between 84° C. and 86° C.; optionally wherein the reagent is at pH 7.45 at 70 C and/or is at a pH of 7.0 at 95° C.;
(d) wherein the reagent comprises at least two different polymerase enzymes
(e) the reagent comprises a polymerase with RNA dependent DNA polymerase activity and a separate polymerase with DNA dependent DNA polymerase activity; and/or
(f) the reagent comprises a polymerase that has both RNA dependent DNA polymerase activity and DNA dependent DNA polymerase activity, optionally wherein the polymerase is selected from the group consisting of: i) SEQ ID NO: 3]; ii) SEQ ID NO: 4]; iii) SEQ ID NO:2 iv) a polymerase with at least 90%, 91%, 92%, 93%, 94%, 95%, 96%, 97%, 98%, 99% or 100% sequence identity to SEQ ID NO: 1, 2, 3, 4, optionally wherein the polymerase comprises 1, 2, 3, 4, 5, 6, 7 or 8 of the following mutations: S515R, I638F, M747K, L322M, L459M, S739G, E773G, and L789F with regard to SEQ ID NO: 1, 2, 3 or 4; v) a polymerase with at least 90%, 91%, 92%, 93%, 94%, 95%, 96%, 97%, 98%, 99% or 100% sequence identity to M1/M747K enzyme [SEQ ID NO: 2].

21. (canceled)

22. (canceled)

23. (canceled)

24. The method of claim 1 wherein: (a) the polymerase has RNA dependent DNA polymerase activity, optionally is Bioneer Rocketscript; or (b) the polymerase has DNA dependent DNA polymerase activity, optionally is tth or tli (Vent) polymerase.

25. (canceled)

26. (canceled)

27. (canceled)

28. The method according to claim 1 wherein: (a) the RNA dependent DNA polymerase activity and the DNA dependent DNA polymerase activity both require the same cofactor, optionally both require magnesium cations as a cofactor; (b) the virus is an RNA virus, the polymerase has RNA dependent DNA polymerase activity, optionally has RNA dependent DNA polymerase activity and DNA dependent DNA polymerase activity; (c) the sample is heated to: i) between around 70° C.-100° C., optionally between 72° C. and 98° C., 74° C. and 96° C., 76° C. and 94° C., 78° C. and 92° C., 80° C. and 90° C., 82° C. and 88° C., or to between 84° C. and 86° C.; and/or to ii) at least 72° C., 74° C., 76° C., 78° C., 80° C., 82° C., 84° C., 96° C., 88° C., 90° C., 92° C., 94° C., 95° C., 96° C., 97° C., 98° C., 99° C., 100° C. or more than 100° C.; and optionally, the sample is heated to the required temperature for: i) between 0.5 s and 5 s, 1 s and 4.5 s, 1.5 s and 4 s, 2 s and 3.5 s, or between 2.5 s and 3 s; and/or ii) at least 0.5 s, 1 s, 1.5 s, 2 s, 2.5 s, 3 s, 3.5 s, 4 s, 4.5 s or at least 5 s; and/or iii) less than 5 s, 4.5 s, 4 s, 3.5 s, 3 s, 2.5 s, 2 s, 1.5 s, 1 s, 0.5 s, 0.25 s.

29. (canceled)

30. (canceled)

31. (canceled)

32. The method of claim 1 wherein the sample is heated to a) between 74° C. and 84° C., optionally for 1 second; or b) more than 90° C. for 1 second; and/or (i) the sample comprises at least 5% of total volume of the sample and reagent, optionally at least 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% or 35% or greater; optionally comprises 13% of total volume of the sample and reagent; (ii) the method is field based; or (c) the sample has not previously been processed, optionally wherein the sample has been taken directly from the subject and directly added to the vessel; and/or the reagent comprises an agent that aids in blood coagulation.

33. (canceled)

34. (canceled)

35. (canceled)

36. (canceled)

37. A method of amplifying microbial or viral nucleic acid present in a sample obtained from a subject wherein the sample is prepared according to claim 1 followed by amplification of the microbial or viral nucleic acid.

38. The method according to claim 37 wherein: (a) the amplification is performed using PCR; (b) the amplification involves a reverse transcription step before PCR, optionally reverse transcription PCR, optionally real time reverse transcription PCR (RT-qPCR); and/or (c) the amplification involves more than one reverse transcription step, optionally 2, 3, 4, 5, 6, 7, 8, 9, 10, 11, 12, 13, 14, 15, 16, 17, 18, 19, 20 or more than 20 steps of reverse transcription before PCR.

39. (canceled)

40. (canceled)

41. The method according to claim 37 wherein the forward primer that drives the reverse transcription is the same as the forward primer that drives the PCR; and optionally, wherein the melting point of the forward primer driving the reverse transcription is different to the melting point of the forward primer driving PCR so that depending on the temperature of the reaction a) reverse transcription occurs, b) PCR occurs, or c) both reverse transcription and PCR occurs simultaneously, optionally wherein the difference in the melting points of the primer is around 5° C.

42. (canceled)

43. The method according to claim 37 wherein: (a) one or more primers comprise one or more LNA, ZNA and/or BNA modifications, optionally wherein the reverse primer comprises one or more LNA, ZNA and/or BNA modifications; (b) the temperature of the reverse transcription reaction is below the melting temperature of the DNA/RNA hybrid; (c) the amplification results in an amplicon with a length of between 40 bp and 500 bp, optionally between 50 bp and 450 bp, optionally between 60 bp and 400 bp, optionally between 70 bp and 350 bp, optionally between 80 bp and 300 bp, optionally between 90 bp and 250 bp, optionally between 100 bp and 200 bp, optionally around 150 bp, optionally between 60 bp to 100 bp; (d) the amplification is performed in the same vessel as that in which the sample has been prepared; (e) wherein once the sample and reagent are in the vessel, the vessel is sealed and is not opened again throughout the preparation and amplification reaction; and/or (f) wherein once the sample and reagent are in the vessel no part of the sample or reagent is removed from the vessel: i) prior to said heating; and/or ii) following said heating; iii) prior to RT; and/or
iv) prior to PCR; and/or v) during RT; and/or vi) during PCR; and/or vii) following RT; and/or viii) following PCR.

44. (canceled)

45. (canceled)

46. (canceled)

47. (canceled)

48. (canceled)

49. The method according to claim 38 wherein: (a) the amplification is performed using fluorophore labelled primers or probes, optionally wherein the fluorophore excites at a wavelength of between 300 nm and 800 nm and emits at a wavelength of between 300 nm and 800 nm, optionally wherein the fluorophore is selected from one or more of FAM, TET, JOE, VIC, HEX, NED, PET, ROX, TAMRA, CY5; and/or (b) the sample and reagent is not manipulated between the reverse transcriptase step and the PCR step, optionally is a One step RT-PCR.

50. (canceled)

51. A method of detecting the presence of a microbe or viral particle in a sample obtained from a subject wherein the sample has been prepared according claim 1 or wherein the nucleic acid from the microbe or viral particle is amplified followed by detection of the amplified nucleic acid; and optionally, wherein the amplification results in a fluorescent signal that corresponds to the quantity of amplicon and/or the detection is performed with a spectrophotometer.

52. (canceled)

53. (canceled)

54. A reagent for use in the preparation of a sample according to claim 1, in a method of detection wherein the reagent comprises a detergent, a solvent and one or more nucleic acid polymerases.

55. The reagent according to claim 54 wherein solvent is glycerol and/or the detergent is Tween, optionally Tween 20, optionally comprises Tween, optionally Tween20 at a concentration of up to 0.4% and/or comprises glycerol at a concentration of up to 11%, optionally wherein the reagent comprises 11%, 10%, 9%, 8%, 7%, 6%, 5%, 4%, 3%, 2%, 1%, 0.5% or less glycerol and/or wherein: (a) the reagent minimises RNA degradation; (b) the reagent does not comprise Tris; (c) the reagent comprises bicine, optionally between 20 mM and 70 mM Bicine, optionally between 25 mM and 65 mM Bicine, optionally between 30 mM and 60 mM Bicine, optionally between 35 mM and 55 mM Bicine, optionally between 40 mM and 50 mM Bicine, optionally 50 mM Bicine; (d) the reagent buffers divalent cations; (e) the reagent comprises at least two different polymerase enzymes.

56. (canceled)

57. (canceled)

58. (canceled)

59. (canceled)

60. (canceled)

61. The reagent according to claim 55 wherein:
(a) the polymerase has DNA dependent DNA polymerase activity; RNA dependent polymerase activity; or has both RNA dependent DNA polymerase activity and DNA dependent DNA polymerase activity, optionally wherein the polymerase is selected from the group consisting of:
i) [SEQ ID NO:1]
ii) Hawk Z05 (Roche), [SEQ ID NO: 4]
iii) SEQ ID NO:2
iv) a polymerase with at least 90%, 91%, 92%, 93%, 94%, 95%, 96%, 97%, 98%, 99% or 100% sequence identity to SEQ ID NO: 1, 2, 3, 4, optionally wherein the polymerase comprises 1, 2, 3, 4, 5, 6, 7 or 8 of the following mutations: S515R, I638F, M747K, L322M, L459M, S739G, E773G, and L789F with regard to SEQ ID NO: 1, 2, 3 or 4;
v) a polymerase with at least 90%, 91%, 92%, 93%, 94%, 95%, 96%, 97%, 98%, 99% or 100% sequence identity to M1/M747K enzyme [SEQ ID NO: 2];
(b) the reagent comprises an agent that aids in blood coagulation;
(c) the reagent comprises components necessary for PCR and/or RT-PCR and or RT-qPCR, optionally comprises one or more primers, optionally one or more fluorophore labelled primers; and/or one or more fluorescent dyes; magnesium chloride; BSA; dNTP; arginine; random RNA e.g. yeast RNA; and/or excipients needed to maintain enzyme activity whilst lyophilised; and optionally,
wherein one or more of the components of the reagent is in lyophilised form, optionally in lyophilised form in a vessel, optionally where one or more of the polymerase, BSA, primer(s), probe(s) or dNTPs are lyophilised, optionally are lyophilised together.

62. (canceled)

63. (canceled)

64. (canceled)

65. A method of performing RT-PCR wherein the method comprises more than one reverse transcription step, optionally 2, 3, 4, 5, 6, 7, 8, 9, 10, 11, 12, 13, 14, 15, 16, 17, 18, 19, 20 or more than 20 steps of reverse transcription prior to or during PCR.

66. The method of claim 65 wherein: (a) the reverse primer that drives the reverse transcription is the same as the reverse primer that drives the PCR; (b) the melting point of the forward primer driving the reverse transcription is different to the melting point of the forward primer driving PCR so that depending on the temperature of the reaction a) reverse transcription occurs, b) PCR occurs, or c) both reverse transcription and PCR occurs simultaneously, optionally wherein the difference in the melting points of the primer is around 5 C; (c) one or more primers comprise one or more LNA, ZNA and/or BNA, modifications, optionally wherein the reverse primer comprises one or more LNA, ZNA and/or BNA modifications; and/or (d) the temperature of the reverse transcription reaction exceeds the melting temperature of the DNA/RNA hybrid; (e) the amplification results in an amplicon with a length of between 40 bp and 500 bp, optionally between 50 bp and 450 bp, optionally between 60 bp and 400 bp, optionally between 70 bp and 350 bp, optionally between 80 bp and 300 bp, optionally between 90 bp and 250 bp, optionally between 100 bp and 200 bp, optionally around 150 bp.

67. (canceled)

68. (canceled)

69. (canceled)

70. (canceled)

71. (canceled)

72. (canceled)

73. (canceled)
